(19) United States
(12) Patent Application Publication
(10) Pub. No.: US 2025/0255510 A1
(43) Pub. Date: Aug. 14, 2025

(54) PROCESSING DEVICE, ENDOSCOPE DEVICE, AND PROCESSING METHOD

(71) Applicant: FUJIFILM Corporation, Tokyo (JP)

(72) Inventors: Seiya TAKENOUCHI, Ashigarakami-gun (JP); Satoru TSUTO, Ashigarakami-gun (JP)

(73) Assignee: FUJIFILM Corporation, Tokyo (JP)

(21) Appl. No.: 19/192,246

(22) Filed: Apr. 28, 2025

Related U.S. Application Data
(63) Continuation of application No. PCT/JP2023/038545, filed on Oct. 25, 2023.

(30) Foreign Application Priority Data
Oct. 31, 2022 (JP) 2022-174971

(51) Int. Cl. A61B 5/06; A61B 1/00

(52) U.S. Cl. CPC A61B 5/062; A61B 1/00009; A61B 1/00071; A61B 1/00158

(57) ABSTRACT

A processing device includes a processor configured to: acquire a distance from a reference position on a movement path of an endoscope to a distal end of the endoscope that is moved along the movement path; acquire a captured image that is captured by the endoscope; and determine a reaching site of the distal end of the endoscope inserted into a subject, based on the captured image and the distance.

### CROSS REFERENCE TO RELATED APPLICATION

This is a continuation of International Application No. PCT/JP2023/038545 filed on Oct. 25, 2023, and claims priority from Japanese Patent Application No. 2022-174971 filed on Oct. 31, 2022, the entire disclosures of which are incorporated herein by reference.

### BACKGROUND OF THE INVENTION

### 1. Field of the Invention

The present invention relates to a processing device, an endoscope device, and a processing method.

### 2. Description of the Related Art

WO2018/211674A discloses an image processing apparatus comprising an acquisition unit that acquires information including an image captured by an endoscope, and a technique level evaluation value calculation unit that calculates a technique level evaluation value indicating a technique level of an operator who operates the endoscope based on the information, in which the technique level evaluation value calculation unit includes a specific scene determination unit that determines a specific scene captured in the image, and an image recording unit that adds identification information for identifying the image to the image in which the specific scene determined by the specific scene determination unit is captured, and records the image.

### SUMMARY OF THE INVENTION

In the present disclosure, a technique capable of determining a position of an endoscope in a subject with high accuracy is provided.

A processing device according to an aspect of the present disclosure comprises: a processor configured to: acquire a distance from a reference position on a movement path of an endoscope to a distal end of the endoscope that is moved along the movement path; acquire a captured image that is captured by the endoscope; and determine a reaching site of the distal end of the endoscope inserted into a subject, based on the captured image and the distance.

An endoscope device according to an aspect of the present disclosure comprises: the processing device and the endoscope.

A processing method according to an aspect of the present disclosure comprises: acquiring a distance from a reference position on a movement path of an endoscope to a distal end of the endoscope that is moved along the movement path; acquiring a captured image that is captured by the endoscope; and determining a reaching site of the distal end of the endoscope inserted into a subject, based on the captured image and the distance.

According to the present disclosure, it is possible to determine the position of the endoscope in the subject with high accuracy.

### BRIEF DESCRIPTION OF THE DRAWINGS

FIG. 1 is a diagram illustrating a schematic configuration of an endoscope system 200.

FIG. 2 is a partial cross-sectional view illustrating a detailed configuration of a soft portion 10A of an endoscope 1.

FIG. 3 is a schematic diagram illustrating details of a magnetic pattern formed on a tubular member 17.

FIG. 4 is a schematic cross-sectional view taken along each of an A-A arrow and a B-B arrow in FIG. 3.

FIG. 5 is an exploded perspective view illustrating a configuration example of a detection unit 40.

FIG. 6 is a schematic diagram of a body part 42A of the detection unit 40 illustrated in FIG. 5 as viewed from a direction x.

FIG. 7 is a diagram illustrating an example of a position at which an insertion part 10 can be located in a through-hole 41.

FIG. 8 is a schematic diagram illustrating an example of a magnetic flux density detected by a magnetic detection unit 43.

FIG. 9 is a schematic diagram illustrating an example of a result of classifying the magnetic flux density illustrated in FIG. 8 according to magnitude thereof.

FIG. 10 is a schematic diagram illustrating another example of the result of classifying the magnetic flux density illustrated in FIG. 8 according to the magnitude thereof.

FIG. 11 is a schematic cross-sectional view illustrating a modification example of magnetic pole portions MA1 and MA2 illustrated in FIG. 3 taken along the A-A arrow and the B-B arrow.

FIG. 12 is a diagram schematically illustrating a magnetic flux line generated in the magnetic pole portion MA1 having the configuration illustrated in FIG. 11.

FIG. 13 is a schematic diagram illustrating a movement path of the insertion part 10 in an examination performed using the endoscope 1.

FIG. 14 is a schematic diagram for describing a first determination example of a reaching site.

FIG. 15 is a schematic diagram for describing a second determination example of the reaching site.

FIG. 16 is a schematic diagram for describing a third determination example of the reaching site.

FIG. 17 is a graph illustrating a display example of examination data associated and recorded by a processor 8P.

### DESCRIPTION OF THE PREFERRED EMBODIMENTS

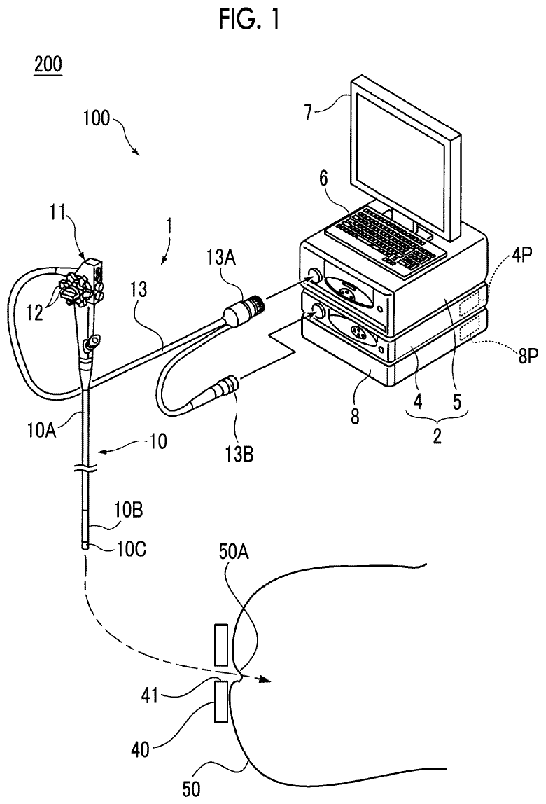

FIG. 1 is a diagram illustrating a schematic configuration of an endoscope system 200. The endoscope system 200 includes an endoscope device 100 having an endoscope 1 as an example of medical equipment that is used by being inserted into a body for examination, surgery, and the like, and a detection unit 40.

The endoscope 1 includes: an insertion part 10 which is an elongated instrument extending in one direction and is inserted into the body; an operating part 11 which is provided in a base end part of the insertion part 10 and is provided with an operation member for performing an observation mode switching operation, an imaging recording operation, a forceps operation, an air supply and water supply operation, a suction operation, an electric cautery operation, or the like; an angle knob 12 provided adjacent to the operating part 11; and a universal cord 13 including connector portions 13A and 13B that respectively connect the endoscope 1 to a light source device 5 and a processor device 4 in an attachable and detachable manner.

The operating part 11 is provided with a forceps port into which biopsy forceps as a treatment tool for collecting a biological tissue such as a cell or a polyp are inserted. It should be noted that, although the illustration is omitted in FIG. 1, various channels such as a forceps channel through which the biopsy forceps inserted from the forceps port are inserted, a channel for air supply and water supply, a channel for suction are provided inside the operating part 11 and the insertion part 10.

The insertion part 10 includes a soft portion 10A having flexibility, a bendable part 10B provided at a distal end of the soft portion 10A, and a distal end part 10C that is provided at a distal end of the bendable part 10B, and is harder than the soft portion 10A. An imaging element and an imaging optical system are built in the distal end part 10C.

The bendable part 10B is configured to be bendable by a rotational movement operation of the angle knob 12. Depending on a site or the like of a subject in which the endoscope 1 is used, the bendable part 10B can be bent in any direction and at any angle, and the distal end part 10C can be directed in a desired direction.

Hereinafter, a direction in which the insertion part 10 extends will be referred to as a longitudinal direction X. Further, one of radial directions of the insertion part 10 will be referred to as a radial direction Y. In addition, one of circumferential directions of the insertion part 10 (one of tangential directions of an outer peripheral edge of the insertion part 10) will be referred to as a circumferential direction Z. In the longitudinal direction X, a direction from a base end (operating part 11 side) of the endoscope 1 toward a distal end will be referred to as a longitudinal direction X1, and a direction from the distal end of the endoscope 1 to the base end will be referred to as a longitudinal direction X2. In addition, in the radial direction Y, one side will be referred to as a radial direction Y1, and the other side will be referred to as a radial direction Y2. The longitudinal direction X is one of directions different from the radial direction Y and the circumferential direction Z. The radial direction Y is one of directions different from the longitudinal direction X and the circumferential direction Z. In the present specification, the longitudinal direction X constitutes a first direction. Further, the radial direction Y constitutes a second direction intersecting the first direction. Further, the circumferential direction Z constitutes a third direction different from the first direction and the second direction.

In the example of FIG. 1, the insertion part 10 of the endoscope 1 is inserted into the body of a subject 50 from an anus 50A of the subject 50. The detection unit 40 has a rectangular plate shape as an example, and has a through-hole 41 into which the insertion part 10 can be inserted. The detection unit 40 is disposed between buttocks of the subject 50 and the insertion part 10 (that is, a movement path of the insertion part 10). The insertion part 10 reaches the anus 50A through the through-hole 41 of the detection unit 40, and is inserted into the body of the subject 50 from the anus 50A. In the present specification, the insertion part 10 constitutes an elongated instrument that is used by being relatively moved with respect to the detection unit 40.

The endoscope device 100 includes: the endoscope 1; a body part 2 consisting of the processor device 4 and the light source device 5 to which the endoscope 1 is connected; a display device 7 that displays a captured image and the like; an input unit 6 that is an interface for inputting various kinds of information to the processor device 4; and an expansion device 8 for expanding various functions.

The processor device 4 has various processors 4P that control the endoscope 1, the light source device 5, and the display device 7. The expansion device 8 has a processor 8P that performs various kinds of processing. Each of the processor 4P and the processor 8P is a central processing unit (CPU) as a general-purpose processor that executes software (a program including a display control program) to perform various functions, a programmable logic device (PLD) as a processor of which a circuit configuration can be changed after the manufacture, such as a field programmable gate array (FPGA), and a dedicated electric circuit as a processor having a circuit configuration specially designed for executing specific processing, such as an application specific integrated circuit (ASIC). Each of the processor 4P and the processor 8P may be composed of one processor, or composed of a combination of two or more processors of the same type or a different type (for example, a combination of a plurality of FPGAs or a combination of a CPU and an FPGA). More specifically, the hardware structure of each of the processor 4P and the processor 8P is an electric circuit (circuitry) in which circuit elements such as semiconductor elements are combined.

The expansion device 8 includes the processor 8P, a communication interface (an interface for communicating with the processor device 4 and the detection unit 40 to be described later) (not illustrated), and a memory composed of a recording medium such as a random access memory (RAM), a read only memory (ROM), a solid state drive (SSD), or a hard disk drive (HDD), and constitutes a processing device.

The processor 8P may perform lesion recognition processing of acquiring a captured image captured by the endoscope 1 from the processor device 4 and recognizing a lesion region in the captured image, treatment tool recognition processing of recognizing whether or not a treatment tool such as forceps or a needle is included in the captured image, and the like. The lesion recognition processing and the treatment tool recognition processing each constitute recognition processing related to the endoscope examination.

The lesion recognition processing refers to processing for performing detection of the lesion region from the captured image, and identification of the detected lesion region. In the lesion recognition processing, the processing for detecting the lesion region is referred to as detection processing, and the processing for identifying the lesion region is referred to as identification processing. The lesion recognition processing may be processing including at least the detection processing. The detection of the lesion region refers to finding a lesion region suspected of a lesion such as a malignant tumor or a benign tumor (lesion candidate region), from the captured image. The identification of the lesion region refers to identifying the type, nature, and the like of the detected lesion region, such as whether the lesion region detected by the detection processing is malignant or benign, what kind of disease in a case where the lesion region is malignant, or how much the degree of progress of the disease is. For example, both the lesion recognition processing and the treatment tool recognition processing can be executed by a recognition model generated by machine learning (for example, a neural network or a support vector machine) or image analysis on the captured image.

The various kinds of processing described below performed by the processor 8P may be performed by the processor 8P alone, or may be performed by being shared between the processor 8P and another processor. The other processor is, for example, a processor of a server in an examination system in which examination data generated by the endoscope system 200 is recorded, the processor 4P, or the like. Alternatively, various kinds of processing performed by the processor 8P can be performed by the processor 4P.

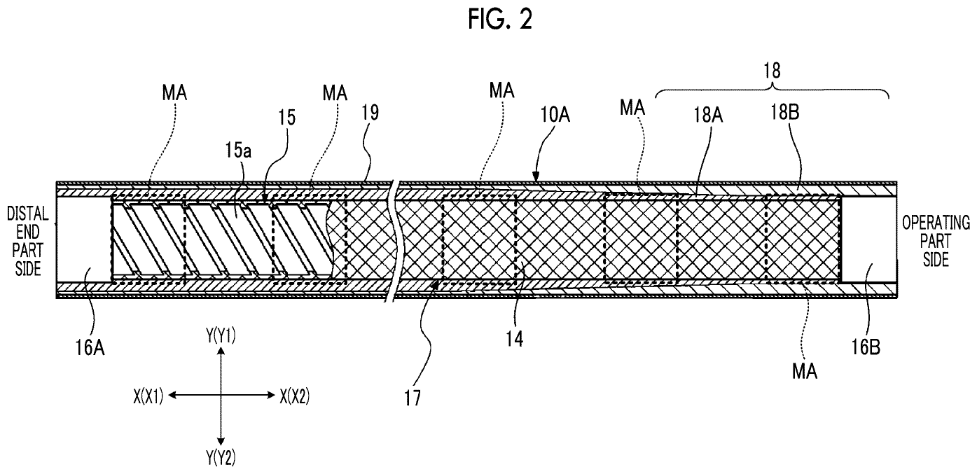

FIG. 2 is a partial cross-sectional view illustrating a detailed configuration of the soft portion 10A of the endoscope 1. The soft portion 10A, which forms most of a length of the insertion part 10, has flexibility over substantially the entire length thereof, and has a structure in which, in particular, a portion to be inserted into a body cavity or the like is rich in flexibility.

The soft portion 10A includes an outer skin layer 18 that constitutes a cylindrical member having an insulating property, and a tubular member 17 that is provided in the outer skin layer 18. The outer skin layer 18 is coated with a coating layer 19.

The tubular member 17 includes: a first member 14 that has a cylindrical shape, contains metal, and is covered with the outer skin layer 18; and a second member 15 that has a cylindrical shape, contains metal, and is inserted into the first member 14. In the example of FIG. 2, the second member 15 is composed of a spiral tube formed by spirally winding a metal strip 15a. Further, the first member 14 is composed of a cylindrical-shaped net body formed by braiding a metal wire. The first member 14 and the second member 15 that continuously extend in the longitudinal direction X and have a thin structure are formed by plastic processing, and the metal constituting these members includes austenitic stainless steel. The austenitic stainless steel cannot be magnetized in a state in which the plastic processing is not performed, but can be magnetized by performing the plastic processing. As described above, each of the first member 14 and the second member 15 constitutes a member that extends in the longitudinal direction X and contains metal.

The outer skin layer 18 is composed of, for example, a resin such as an elastomer, and has a multi-layer structure of an inner resin layer 18A and an outer resin layer 18B. The outer skin layer 18 may have a monolayer structure. In the first member 14 and the second member 15, a cap 16A is fitted to an end part on the distal end part 10C side, and a cap 16B is fitted to an end part on the operating part 11 side. The cap 16A and the cap 16B are covered with the outer skin layer 18. The soft portion 10A is connected to the bendable part 10B at the cap 16A, and is connected to the operating part 11 at the cap 16B.

The tubular member 17 of the soft portion 10A is formed with a magnetic pattern along the longitudinal direction X. The magnetic pattern along the longitudinal direction X refers to a pattern in which two types of magnetic pole regions, which are a negative pole (S pole) and a positive pole (N pole), are arranged in a predetermined arrangement pattern in the longitudinal direction X. As illustrated in FIG. 2, each of the first member 14 and the second member 15 is provided with a plurality of magnetic pole portions MA including the magnetic pole region. At least one of the two types of magnetic pole regions, which are the negative pole (S pole) and the positive pole (N pole), is formed on the magnetic pole portion MA. As described above, each of the first member 14 and the second member 15 constitutes the member that extends in the longitudinal direction X and has the magnetic pattern formed along the longitudinal direction X.

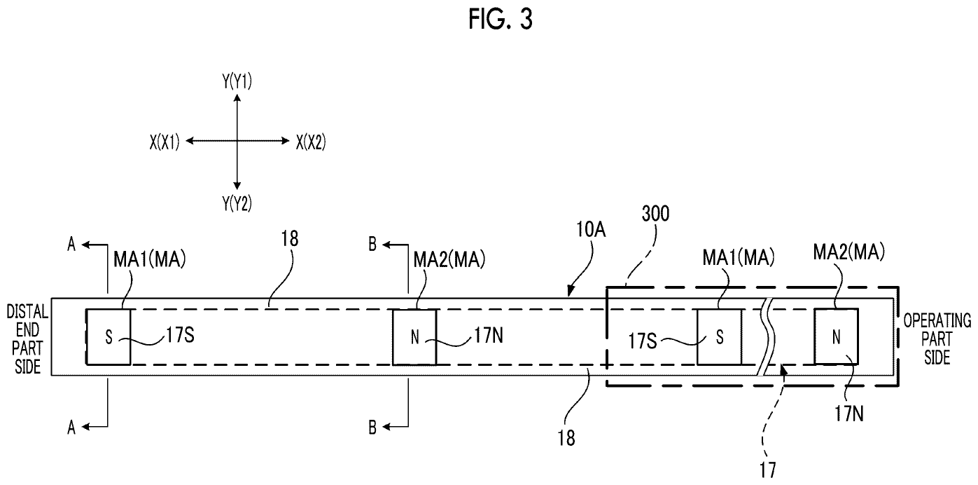

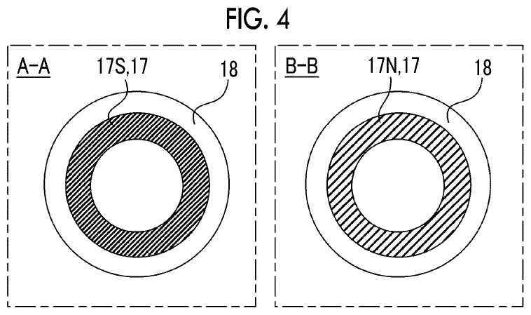

FIG. 3 is a schematic diagram illustrating details of the magnetic pattern formed on the tubular member 17. FIG. 4 is a schematic cross-sectional view taken along each of an A-A arrow and a B-B arrow in FIG. 3. As illustrated in FIGS. 3 and 4, in the tubular member 17, a magnetic pole portion MA1 including a negative pole region 17S formed in an annular shape along the circumferential direction of the tubular member 17, and a magnetic pole portion MA2 including a positive pole region 17N formed in an annular shape along the circumferential direction of the tubular member 17 are provided to be alternately arranged in the longitudinal direction X. The total number of the magnetic pole portions MA1 and the total number of the magnetic pole portions MA2 are the same.

Here, an example of a manufacturing method of the endoscope 1 including the tubular member 17 having the magnetic pattern illustrated in FIG. 3 will be described. First, the endoscope 1 having the configuration illustrated in FIG. 1 is manufactured by a well-known method. Next, a magnetic field generation device 300 is prepared, which has a cylindrical coil, and can generate a magnetic field in the cylindrical coil by allowing a current to flow through the cylindrical coil. Next, as illustrated in FIG. 3, the insertion part 10 of the endoscope 1 is inserted into the cylindrical coil of the magnetic field generation device 300 from the distal end side to relatively move the coil to a boundary portion between the operating part 11 and the soft portion 10A. In this state, a step of allowing an alternating current to flow through the cylindrical coil of the magnetic field generation device 300 to form a magnetic field, and pulling out the insertion part 10 from the cylindrical coil of the magnetic field generation device 300 in the longitudinal direction X2 at a constant speed is performed. In this step, a magnetic force of the tubular member 17 generated by the plastic processing is removed, and the tubular member 17 is demagnetized. In this step, it is preferable to pull out the insertion part 10 until the bendable part 10B and the distal end part 10C pass through the cylindrical coil, and to demagnetize the entire insertion part 10. That is, in the insertion part 10 of the endoscope 1, it is preferable that the bendable part 10B and the distal end part 10C are demagnetized. The demagnetization of a certain region means that a magnetic flux density detected from the region is equal to or less than the geomagnetism.

After the demagnetization of at least the tubular member 17 (soft portion 10A) is performed, a work of forming a state in which the cylindrical coil of the magnetic field generation device 300 is disposed on an outer periphery of the soft portion 10A at a predetermined position in the longitudinal direction X, and allowing the alternating current to flow through the cylindrical coil in that state to form the magnetic field is performed. By this work, the negative pole region 17S and the positive pole region 17N are formed over the entire circumferential direction of the tubular member 17 at positions in the vicinity of both ends of the cylindrical coil of the magnetic field generation device 300. Thereafter, by repeating this work while shifting the position of the soft portion 10A with respect to the cylindrical coil in the longitudinal direction X, the magnetic pattern illustrated in FIG. 3 can be formed on the tubular member 17.

By adopting such a manufacturing method, any magnetic pattern can be easily formed on the tubular member 17 of the soft portion 10A even in the endoscope 1 having the existing configuration or the endoscope 1 that has already been sold. In addition, by performing the demagnetization of the tubular member 17 of the soft portion 10A and then forming the magnetic pattern on the tubular member 17, the magnetic pattern having a desired magnetic force can be formed with high accuracy. Further, by forming the magnetic pole region by using the cylindrical coil, it is possible to form the magnetic pole region having a uniform magnetic force (magnetic flux density) over the entire outer periphery of the tubular member 17 in the magnetic pole portion MA. In FIG. 3, a boundary line between each of the negative pole region 17S and the positive pole region 17N, and the other region in the tubular member 17 is illustrated, but this boundary line is illustrated for convenience, and is invisible. It is preferable that information on the magnetic pattern formed on the tubular member 17 is recorded in a memory (for example, a memory provided in the expansion device 8) accessible by the processor 8P. The information on the magnetic pattern includes information indicating positions of the two types of magnetic pole regions in the tubular member 17, information indicating an arrangement pitch of the two types of magnetic pole regions in the tubular member 17, information indicating a range in which the magnetic pole region is formed on the insertion part 10, information indicating the position of the demagnetized region in the insertion part 10, or the like. The demagnetized region in the insertion part 10 constitutes an adjacent region adjacent to the region in which the magnetic pattern is formed on the insertion part 10. The bendable part 10B and the distal end part 10C are demagnetized regions in the insertion part 10, but the bendable part 10B and the distal end part 10C need only be configured to be distinguishable from the region in which the magnetic pattern is formed, and it is not essential that the bendable part 10B and the distal end part 10C are demagnetized. For example, magnetization may be performed with a pattern or a magnetic force that is clearly different from the magnetic pattern.

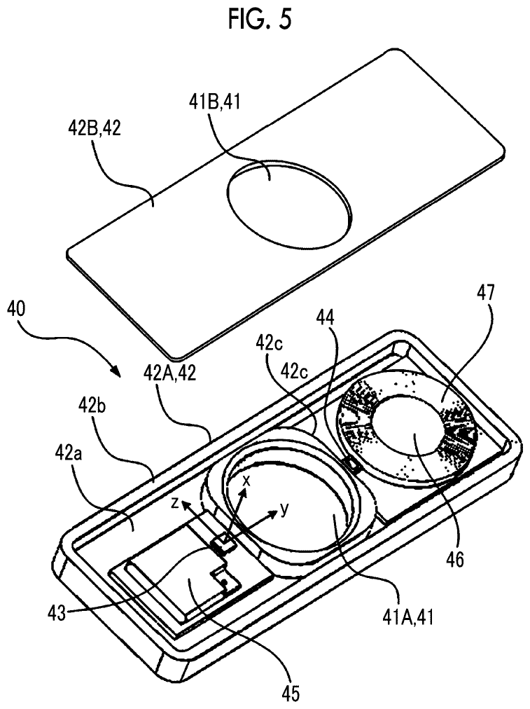

FIG. 5 is an exploded perspective view illustrating a configuration example of the detection unit 40. The detection unit 40 includes a housing 42 having the through-hole 41; and a magnetic detection unit 43, a magnetic detection unit 44, a communication chip 45, a storage battery 46, and a power receiving coil 47 that are accommodated in the housing 42.

The housing 42 includes: a body part 42A including a flat plate portion 42a that has a rectangular flat plate shape and has a through-hole 41A penetrating in a thickness direction, a side wall portion 42b that has a rectangular frame shape of rising from an outer peripheral edge portion of the flat plate portion 42a in the thickness direction of the flat plate portion 42a, and an inner wall portion 42c that has a cylindrical shape of rising from a peripheral edge portion of the through-hole 41A in the flat plate portion 42a in the thickness direction of the flat plate portion 42a; and a lid portion 42B that has a rectangular flat plate shape for closing an accommodation space surrounded by the flat plate portion 42a, the side wall portion 42b, and the inner wall portion 42c. The magnetic detection unit 43, the magnetic detection unit 44, the communication chip 45, the storage battery 46, and the power receiving coil 47 are accommodated in this accommodation space.

A through-hole 41B penetrating in the thickness direction is formed on the lid portion 42B, and in a state in which the lid portion 42B closes the accommodation space, the through-hole 41A and the through-hole 41B communicate with each other through an inner peripheral portion of the inner wall portion 42c to form the through-hole 41 into which the endoscope 1 can be inserted. It is preferable that the through-hole 41 has a perfect circular shape as viewed from an axial direction of the inner wall portion 42c (direction in which the endoscope 1 is inserted). The housing 42 is preferably composed of a resin or the like in order to reduce the weight and the cost, and preferably has a structure that prevents moisture from entering the accommodation space.

Each of the magnetic detection unit 43 and the magnetic detection unit 44 is disposed close to the inner wall portion 42c, and is a three-axis magnetic sensor that can detect a magnetic flux density in a direction x (direction along the axis of the through-hole 41) along the axis of the inner wall portion 42c, a magnetic flux density in a radial direction y of the through-hole 41, and a magnetic flux density in a direction z orthogonal to the direction x and the radial direction y.

In a state in which the insertion part 10 of the endoscope 1 is inserted into the through-hole 41, the longitudinal direction X of the insertion part 10 and the direction x match each other, the radial direction Y of the insertion part 10 and the radial direction y match each other, and the circumferential direction Z of the insertion part 10 and the direction z match each other. Therefore, each of the magnetic detection unit 43 and the magnetic detection unit 44 is configured to detect a magnetic flux density BX in the longitudinal direction X of the insertion part 10, a magnetic flux density BY in the radial direction Y of the insertion part 10, and a magnetic flux density BZ in the circumferential direction Z of the insertion part 10. Each of the magnetic detection unit 43 and the magnetic detection unit 44 may include three magnetic sensors, which are a uniaxial magnetic sensor that can detect the magnetic flux density BX, a uniaxial magnetic sensor that can detect the magnetic flux density BY, and a uniaxial magnetic sensor that can detect the magnetic flux density BZ. In the present specification, the magnetic flux density BX constitutes a first magnetic flux density, the magnetic flux density BY constitutes a second magnetic flux density, and the magnetic flux density BZ constitutes a third magnetic flux density.

Each of the magnetic detection unit 43 and the magnetic detection unit 44 need only be able to detect the magnetic flux density including a component in the longitudinal direction X, the magnetic flux density including a component in the radial direction Y, and the magnetic flux density including a component in the circumferential direction Z, and three detection axis directions may not exactly match the longitudinal direction X, the radial direction Y, and the circumferential direction Z, respectively. In the magnetic sensor, in a case in which a first detection axis direction is different from the radial direction Y and the circumferential direction Z, a second detection axis direction is different from the longitudinal direction X and the circumferential direction Z, and a third detection axis direction is different from the radial direction Y and the longitudinal direction X, the magnetic sensor can detect the magnetic flux density including the component in the longitudinal direction X, can detect the magnetic flux density including the component in the radial direction Y, and can detect the magnetic flux density including the component in the circumferential direction Z.

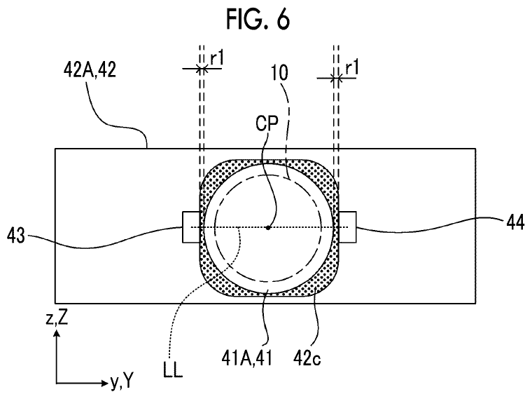

FIG. 6 is a schematic diagram of the body part 42A of the detection unit 40 illustrated in FIG. 5 as viewed from the direction x. As illustrated in FIG. 6, the magnetic detection unit 43 and the magnetic detection unit 44 are disposed at positions facing each other with a center CP of the through-hole 41 interposed therebetween as viewed in the direction x. That is, in a state of being viewed in the direction x, a midpoint of a line segment LL connecting the magnetic detection unit 43 and the magnetic detection unit 44 substantially matches the center CP of the through-hole 41. In other words, a distance from the magnetic detection unit 43 to the center CP of the through-hole 41 and a distance from the magnetic detection unit 44 to the center CP of the through-hole 41 substantially match each other.

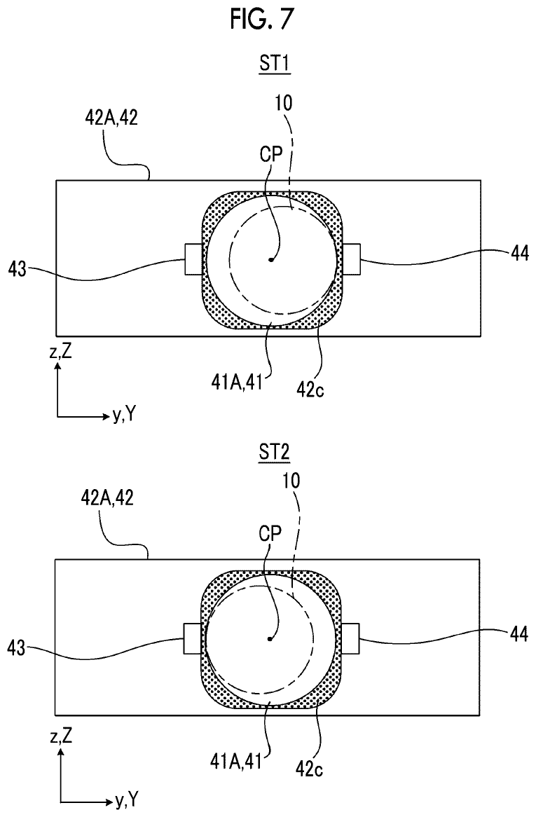

FIG. 7 is a diagram illustrating an example of a position at which the insertion part 10 can be located in the through-hole 41. A state ST1 of FIG. 7 illustrates a state in which the insertion part 10 is most distant from the magnetic detection unit 43 in the radial direction Y in the through-hole 41. A state ST2 of FIG. 7 illustrates a state in which the insertion part 10 is most distant from the magnetic detection unit 44 in the radial direction Y in the through-hole 41. A detection range and an installation position of each of the magnetic detection unit 43 and the magnetic detection unit 44 are determined such that the magnetic flux density can be detected with high accuracy from the magnetic pattern formed on the tubular member 17 in any of the state ST1 and the state ST2 of FIG. 7.

In the present embodiment, as illustrated in FIG. 6, a thickness of a portion of the inner wall portion 42c, the portion being at the same position as the center CP in the direction z, is a thickness r1. The thickness r1 is 0.5 mm, for example. In a case in which the magnetic force of the magnetic pole region formed on the tubular member 17 is defined by the magnetic flux density detected at a position distant from an outer surface of the insertion part 10 in the radial direction of the insertion part 10 by 0.5 mm, it is preferable that the magnetic force has a value that is sufficiently larger than the geomagnetism and is equal to or larger than a value (specifically, 500 microtesla) suitable for the performance of a general magnetic sensor. In addition, for example, in the state ST1 or the state ST2 of FIG. 7, it is more preferable that the magnetic force of the magnetic pole region formed on the tubular member 17 is in a range of 1000 microtesla to 1500 microtesla such that the magnetic detection unit 43 and the magnetic detection unit 44 can detect the magnetic flux density with high accuracy. However, it is preferable that an upper limit value of the magnetic force of the magnetic pole region formed on the tubular member 17 is equal to or less than 20 millitesla such that the insertion part 10 does not adhere to another metal. In consideration of the maximum sensitivity of the general magnetic sensor, it is more preferable that the upper limit value of the magnetic force of the magnetic pole region formed on the tubular member 17 is equal to or less than 2 millitesla.

As illustrated in FIG. 7, the position of the insertion part 10 in the through-hole 41 can be changed. However, by obtaining the arithmetic mean of the magnetic flux density BX detected from the tubular member 17 by the magnetic detection unit 43 and the magnetic flux density BX detected from the tubular member 17 by the magnetic detection unit 44, it is possible to detect the magnetic flux density BX according to the magnetic pattern regardless of the position of the insertion part 10 in the through-hole 41. Similarly, by obtaining the arithmetic mean of the magnetic flux density BY detected from the tubular member 17 by the magnetic detection unit 43 and the magnetic flux density BY detected from the tubular member 17 by the magnetic detection unit 44, it is possible to detect the magnetic flux density BY according to the magnetic pattern regardless of the position of the insertion part 10 in the through-hole 41. Similarly, by obtaining the arithmetic mean of the magnetic flux density BZ detected from the tubular member 17 by the magnetic detection unit 43 and the magnetic flux density BZ detected from the tubular member 17 by the magnetic detection unit 44, it is possible to detect the magnetic flux density BZ according to the magnetic pattern regardless of the position of the insertion part 10 in the through-hole 41.

The communication chip 45 illustrated in FIG. 5 transmits information on the magnetic flux density detected by each of the magnetic detection unit 43 and the magnetic detection unit 44 to the expansion device 8 by wireless communication. In the present specification, the communication chip 45 constitutes an output unit that outputs the information detected by the magnetic detection unit 43 and the magnetic detection unit 44 to the outside. This information on the magnetic flux density may be transmitted to the processor device 4, and in this case, this information is transmitted by the processor 4P to the processor 8P of the expansion device 8.

The storage battery 46 is charged by the power received by the power receiving coil 47 by the noncontact power supply. The magnetic detection unit 43, the magnetic detection unit 44, and the communication chip 45 are operated by the power supplied from the storage battery 46. The detection unit 40 has a start-up switch (not illustrated). By performing an operation to turn on the start-up switch, the power supply from the storage battery 46 to the magnetic detection unit 43, the magnetic detection unit 44, and the communication chip 45 is started. The detection unit 40 may have a configuration in which the start-up switch is not provided and the power supply to the magnetic detection unit 43, the magnetic detection unit 44, and the communication chip 45 is started by receiving wireless power supply from the outside. In a case in which the start-up switch is not provided, a structure in which the accommodation space of the housing 42 is completely sealed can be easily realized.

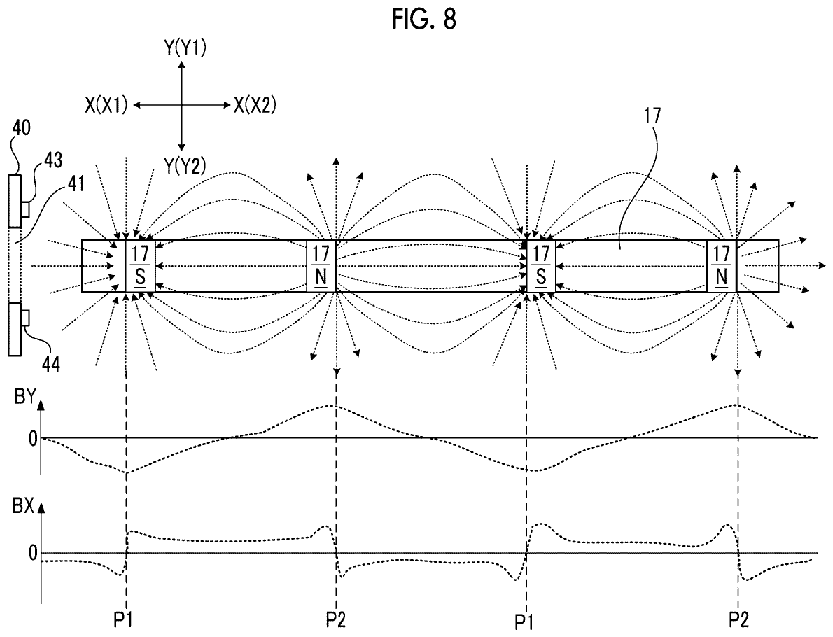

FIG. 8 is a schematic diagram illustrating an example of the magnetic flux density detected by the magnetic detection unit 43. Since the magnetic flux density detected by the magnetic detection unit 44 is the same as that in FIG. 8, the illustration is omitted. Two graphs illustrated in FIG. 8 illustrate the magnetic flux density BX and the magnetic flux density BY that are detected by the magnetic detection unit 43 in a case where the soft portion 10A is moved in the longitudinal direction X1 through the through-hole 41. In FIG. 8, a magnetic flux line from the positive pole region 17N to the negative pole region 17S adjacent to the positive pole region 17N in the longitudinal direction X is indicated by a broken line arrow.

In a case in which the soft portion 10A (tubular member 17) is moved toward the through-hole 41 of the detection unit 40 illustrated in the upper left of FIG. 8, as illustrated in the graph of FIG. 8, the magnetic flux density BX detected by the magnetic detection unit 43 has a positive value between each positive pole region 17N and the negative pole region 17S adjacent to the positive pole region 17N in the longitudinal direction X1, and has a negative value between each positive pole region 17N and the negative pole region 17S adjacent to the positive pole region 17N in the longitudinal direction X2. In addition, the magnetic flux density BY detected by the magnetic detection unit 43 has a negative value and a large absolute value in the vicinity of the negative pole region 17S, has a positive value and a large absolute value in the vicinity of the positive pole region 17N, and has a value close to zero in the vicinity of an intermediate position between the negative pole region 17S and the positive pole region 17N.

Regarding the magnetic flux densities detected from the magnetic pattern formed on the tubular member 17 at a plurality of positions in the longitudinal direction X of the tubular member 17, each of the magnetic flux density BX and the magnetic flux density BY is periodically changed with positive and negative values, and the phases of the magnetic flux density BX and the magnetic flux density BY are shifted from each other in the longitudinal direction X. In the negative pole region 17S, an end (portion of a position P1 in FIG. 8) in the longitudinal direction X where the absolute value of the magnetic flux density BY is the maximum value is hereinafter referred to as a negative pole end. In the positive pole region 17N, an end (portion of a position P2 in FIG. 8) in the longitudinal direction X where the absolute value of the magnetic flux density BY is the maximum value is hereinafter referred to as a positive pole end.

As an example, by magnetizing the tubular member 17 using the method described above by setting a length of the cylindrical coil of the magnetic field generation device 300 in the axial direction to 60 mm, an inner diameter of the cylindrical coil of the magnetic field generation device 300 to 18 mm, and a movement pitch of the cylindrical coil in the longitudinal direction X to 144 mm, it is possible to form the magnetic pattern in which a distance between the negative pole end and the positive pole end is 72 mm. In the example of FIG. 8, for example, by disposing the cylindrical coil between the negative pole region 17S at the left end and the positive pole region 17N adjacent to the right side of the negative pole region 17S to form the magnetic field, it is possible to form these two magnetic pole regions. Then, from that state, by relatively moving the cylindrical coil by 144 mm in the longitudinal direction X2 to form the magnetic field in that state, it is possible to form the positive pole region 17N at the right end and the negative pole region 17S adjacent to the left side of the positive pole region 17N. In this manner, it is possible to form the magnetic pattern in which the distance (distance between the position P1 and the position P2) between the positive pole end and the negative pole end which are alternately formed in the longitudinal direction X is 72 mm.

In the endoscope system 200, the processor 8P of the expansion device 8 acquires the information on the magnetic flux densities detected by the magnetic detection unit 43 and the magnetic detection unit 44, from the detection unit 40, and determines the movement state of the insertion part 10 in the longitudinal direction X on the basis of the acquired magnetic flux density BX and magnetic flux density BY. The movement state of the insertion part 10 determined here includes: a movement direction indicating in which direction in the longitudinal direction X the insertion part 10 is moved with respect to the detection unit 40; and a movement amount (movement distance) indicating how much distance the insertion part 10 inserted into the through-hole 41 of the detection unit 40 has moved in the longitudinal direction X with respect to the detection unit 40. The processor 8P obtains the arithmetic mean of the magnetic flux densities BX respectively detected at the same timing by the magnetic detection unit 43 and the magnetic detection unit 44, obtains the arithmetic mean of the magnetic flux densities BY respectively detected at the same timing by the magnetic detection unit 43 and the magnetic detection unit 44, and determines the movement state of the insertion part 10 on the basis of the magnetic flux density BX and the magnetic flux density BY obtained by these arithmetic means.

The processor 8P classifies the magnetic flux density BX into a plurality of pieces of information according to the magnitude thereof, classifies the magnetic flux density BY into a plurality of pieces of information according to the magnitude thereof, and determines the movement state of the insertion part 10 in the longitudinal direction X on the basis of a combination of any of the plurality of pieces of information obtained by classifying the magnetic flux density BX and any of the plurality of pieces of information obtained by classifying the magnetic flux density BY.

Specifically, the processor 8P sets a first threshold value th (for example, “0”) as a threshold value for classifying the magnetic flux density BX into two levels, and sets a second threshold value th1 (positive value larger than 0) and a second threshold value th2 (negative value less than 0) as a threshold value for classifying the magnetic flux density BY into three levels. Moreover, the processor 8P classifies the magnetic flux density BX by setting a value larger than the first threshold value th as a high level H and setting a value less than the first threshold value th as a low level L. Further, the processor 8P classifies the magnetic flux density BY by setting a value larger than the second threshold value th1 as the high level H, setting a value between the second threshold value th1 and the second threshold value th2 as a middle level M, and setting a value less than the second threshold value th2 as the low level L. The result of classifying the magnetic flux density BX in this manner is also referred to as a classification level of the magnetic flux density BX, and the result of classifying the magnetic flux density BY in this manner is also referred to as a classification level of the magnetic flux density BY. In the present specification, among the classification levels of the magnetic flux density BX, the high level constitutes one of fourth information and fifth information, and the low level constitutes the other of the fourth information and the fifth information. In addition, among the classification levels of the magnetic flux density BY, the high level constitutes one of first information and second information, the low level constitutes the other of the first information and the second information, and the middle level constitutes third information.

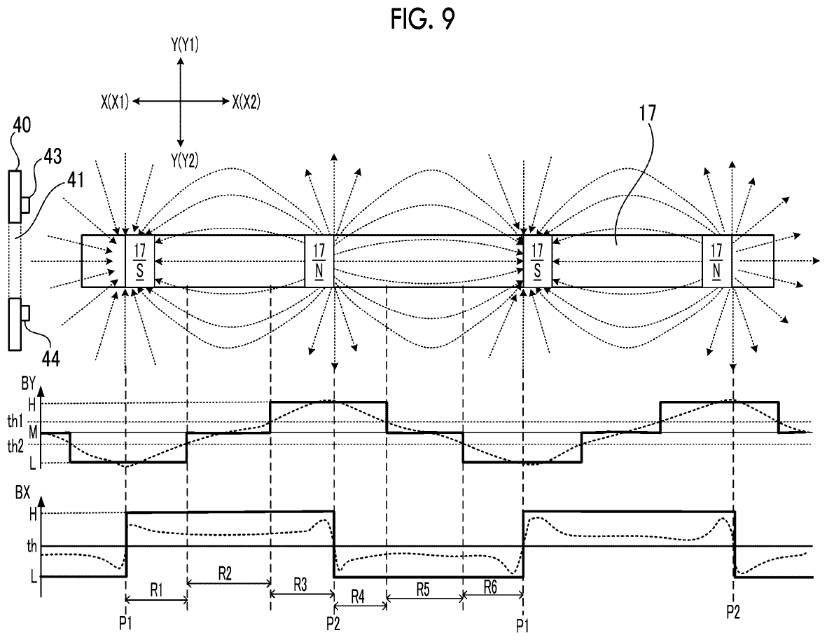

In FIG. 9, the result (classification level) of classifying the magnetic flux density BX and the magnetic flux density BY in the graphs illustrated in FIG. 8 is indicated by a thick solid line. As illustrated in FIG. 9, in the tubular member 17, a range between two adjacent positions P1 (between the negative pole ends) is divided into: a region R1 in which the magnetic flux density BX is at the high level and the magnetic flux density BY is at the low level; a region R2 in which the magnetic flux density BX is at the high level and the magnetic flux density BY is at the middle level; a region R3 in which the magnetic flux density BX is at the high level and the magnetic flux density BY is at the high level; a region R4 in which the magnetic flux density BX is at the low level and the magnetic flux density BY is at the high level; a region R5 in which the magnetic flux density BX is at the low level and the magnetic flux density BY is at the middle level; and a region R6 in which the magnetic flux density BX is at the low level and the magnetic flux density BY is at the low level. As described above, the range between the negative pole ends adjacent to each other in the longitudinal direction X can be divided into six regions R1 to R6 depending on the combination of the classification level of the magnetic flux density BX and the classification level of the magnetic flux density BY.

By monitoring the thick solid lines (classification levels of the magnetic flux densities BX and BY) illustrated in FIG. 9, the processor 8P determines the movement direction of the insertion part 10 with respect to the detection unit 40, and the movement amount (movement distance) of the insertion part 10 in the longitudinal direction X starting from the position of the detection unit 40.

For example, in a case in which the negative pole region 17S provided on the most distal end side of the tubular member 17 passes through the through-hole 41, the processor 8P detects that the region R1 at the most distal end of the tubular member 17 is located in the through-hole 41, from the combination of the classification level of the magnetic flux density BX and the classification level of the magnetic flux density BY, and detects the position as a reference position. The distance (referred to as a distance Li) in the longitudinal direction X from the negative pole region 17S provided on the most distal end side of the tubular member 17 to the distal end of the distal end part 10C is known. Therefore, in a case in which this reference position is detected, the processor 8P determines that the movement distance of the insertion part 10 with respect to the detection unit 40 is “0”, and further determines that an insertion length (distance from the reference position (through-hole 41) to the distal end of the insertion part 10) of the insertion part 10 into the body of the subject 50 is the distance Li.

After the reference position is detected, in a case in which it is determined that the region of the tubular member 17 passing through the through-hole 41 is being changed in a direction from the region R1 to the region R6 according to the classification levels of the magnetic flux densities BX and BY, the processor 8P determines that the insertion part 10 is being moved in the longitudinal direction X1. In addition, in a case in which it is determined that the insertion part 10 is being moved in the longitudinal direction X1, the processor 8P increases the movement distance of the insertion part 10 in the longitudinal direction X1 by a unit distance ΔL and increases the insertion length of the insertion part 10 into the body of the subject 50 by the unit distance ΔL, each time the region of the tubular member 17 passing through the through-hole 41 is changed by one (for example, a change from the region R1 to the region R2 or a change from the region R2 to the region R3). The unit distance ΔL can be a value obtained by dividing an interval between the adjacent negative pole regions 17S by 6.

On the other hand, in a case in which it is determined that the region of the tubular member 17 passing through the through-hole 41 is being changed in a direction from the region R6 to the region R1 according to the classification levels of the magnetic flux densities BX and BY, the processor 8P determines that the insertion part 10 is being moved in the longitudinal direction X2. In addition, in a case in which it is determined that the insertion part 10 is being moved in the longitudinal direction X2, the processor 8P decreases the movement distance of the insertion part 10 in the longitudinal direction X1 by a unit distance ΔL and decreases the insertion length of the insertion part 10 into the body of the subject 50 by the unit distance ΔL, each time the region of the tubular member 17 passing through the through-hole 41 is changed by one.

Depending on the movement speed of the insertion part 10, it can also be determined that the region of the tubular member 17 passing through the through-hole 41 is changed from the region R1 to the region R3 or is changed from the region R3 to the region R1. In a case in which it is determined that the region of the tubular member 17 passing through the through-hole 41 is changed by two in this manner, the processor 8P need only increase or decrease the insertion length of the insertion part 10 by twice the unit distance ΔL.

The processor 8P displays the information on the insertion length determined in this manner on the display device 7, outputs the information by voice from a speaker (not illustrated), or transmits the information to an operator of the endoscope 1 by vibration of a vibrator provided in the operating part 11. As a result, it is possible to accurately record an imaging position by the endoscope 1, guide or evaluate the operation of the endoscope 1, and the like.

As described above, by demagnetizing the distal end part 10C and the bendable part 10B in the insertion part 10, the processor 8P can easily detect the reference position. Specifically, in a case in which the insertion part 10 is inserted into the through-hole 41 from the distal end side and is moved in the longitudinal direction X1, both the magnetic flux density BX and the magnetic flux density BY are values in the vicinity of “0” while the distal end part 10C and the bendable part 10B pass through the through-hole 41. Further, at a point in time at which the negative pole region 17S on the most distal end side of the tubular member 17 reaches the through-hole 41, the magnetic flux density BX and the magnetic flux density BY are a combination of the high level and the low level as illustrated in FIG. 9, and therefore, it is possible to easily detect the reference position by the fluctuation of the magnetic flux density.

As described above, the processor 8P classifies the magnetic flux density BX into two of the high level and the low level, classifies the magnetic flux density BY into three of the high level, the middle level, and the low level, and determines the movement state of the insertion part 10 in the longitudinal direction X on the basis of the combination thereof. In this way, by monitoring the change in the combination of the classification level of the magnetic flux density BX and the classification level of the magnetic flux density BY, the movement direction, the movement distance, and the insertion length of the insertion part 10 can be determined. With the endoscope system 200, such an effect can be realized only by magnetizing the endoscope 1 having a general-purpose configuration and adding the detection unit 40, so that a construction cost of the system can be reduced. In addition, since the movement direction, the movement distance, and the insertion length of the insertion part 10 are determined on the basis of the information on the magnetic flux density that can be acquired non-optically, even in a case in which the insertion part 10 is dirty, the determination accuracy is not reduced, and thus it is practical.

In addition, by using the combination of the classification level of the magnetic flux density BX and the classification level of the magnetic flux density BY, it is possible to determine the movement distance of the insertion part 10 with a resolution (for example, a unit of ⅓ of the interval) finer than the interval between the two types of adjacent magnetic pole regions (negative pole region 17S and positive pole region 17N). In this way, the movement distance can be finely determined, which can be useful for accurate recording of the imaging position by the endoscope 1, guide or evaluation of the operation of the endoscope 1, and the like.

In addition, the processor 8P obtains the arithmetic mean of the magnetic flux density detected by the magnetic detection unit 43 and the magnetic flux density detected by the magnetic detection unit 44, and determines the movement direction, the movement distance, and the insertion length of the insertion part 10 on the basis of the magnetic flux density of the arithmetic mean. Therefore, it is possible to obtain the change in the magnetic flux density according to the magnetic pattern regardless of the position of the insertion part 10 in the through-hole 41. In addition, the magnetic flux densities detected by the magnetic detection unit 43 and the magnetic detection unit 44 can include a disturbance component caused by geomagnetism, a magnetic field generated by a steel frame of a building, a magnetic field generated by the steel furniture, and the like, in addition to a magnetic field generated by magnetization. However, as described above, by obtaining the arithmetic mean of the magnetic flux densities detected by the two magnetic detection units, it is possible to reduce an influence of the disturbance component.

In a case in which a difference between the inner diameter of the through-hole 41 and the outer diameter of the insertion part 10 is made as small as possible, any one of the magnetic detection unit 43 or the magnetic detection unit 44 provided in the detection unit 40 is not essential and can be omitted. In this case, the processor 8P need only determine the movement direction, the movement distance, and the insertion length of the insertion part 10 on the basis of the magnetic flux densities BX and BY detected by the magnetic detection unit 43 or the magnetic detection unit 44.

In addition, in the present embodiment, each of the negative pole region 17S and the positive pole region 17N formed on the tubular member 17 is formed in an annular shape along the outer periphery of the tubular member 17. Therefore, even in a case in which the insertion part 10 is rotated in the circumferential direction thereof in the through-hole 41, it is possible to substantially eliminate the change in the magnetic flux densities detected by the magnetic detection unit 43 and the magnetic detection unit 44. Therefore, the movement direction, the movement distance, and the insertion length of the insertion part 10 can be determined regardless of the posture of the insertion part 10.

The disturbance component can be included in the magnetic flux densities detected by the magnetic detection unit 43 and the magnetic detection unit 44. In addition, the orientation of the disturbance component is also changed depending on the posture of the detection unit 40. Therefore, the influence of the disturbance component can be eliminated by classifying the magnetic flux density BX into two of the high level and the low level, classifying the magnetic flux density BY into three of the high level, the middle level, and the low level, and determining the movement state of the insertion part 10 in the longitudinal direction X on the basis of the combination of the classification levels as described above, rather than determining the movement state of the insertion part 10 in the longitudinal direction X using raw data of the magnetic flux density BX and the magnetic flux density BY as they are.

In the above description, the processor 8P classifies the magnetic flux density BX into two of the high level and the low level, classifies the magnetic flux density BY into three of the high level, the middle level, and the low level, and determines the movement state of the insertion part 10 in the longitudinal direction X on the basis of the combination of the classification levels. As a modification example, the processor 8P may classify the magnetic flux density BX into two of the high level and the low level, may classify the magnetic flux density BY into two of the high level and the low level, and may determine the movement state of the insertion part 10 in the longitudinal direction X on the basis of the combination of the classification levels.

Specifically, the processor 8P sets the “first threshold value th (for example, 0)” as the threshold value for classifying the magnetic flux density BX into two levels, and sets a “second threshold value th3 (for example, 0)” as the threshold value for classifying the magnetic flux density BY into two levels. Moreover, the processor 8P classifies the magnetic flux density BX by setting a value larger than the first threshold value th as the high level and setting a value less than the first threshold value th as the low level. Further, the processor 8P classifies the magnetic flux density BY by setting a value larger than the second threshold value th3 as the high level and setting a value less than the second threshold value th3 as the low level.

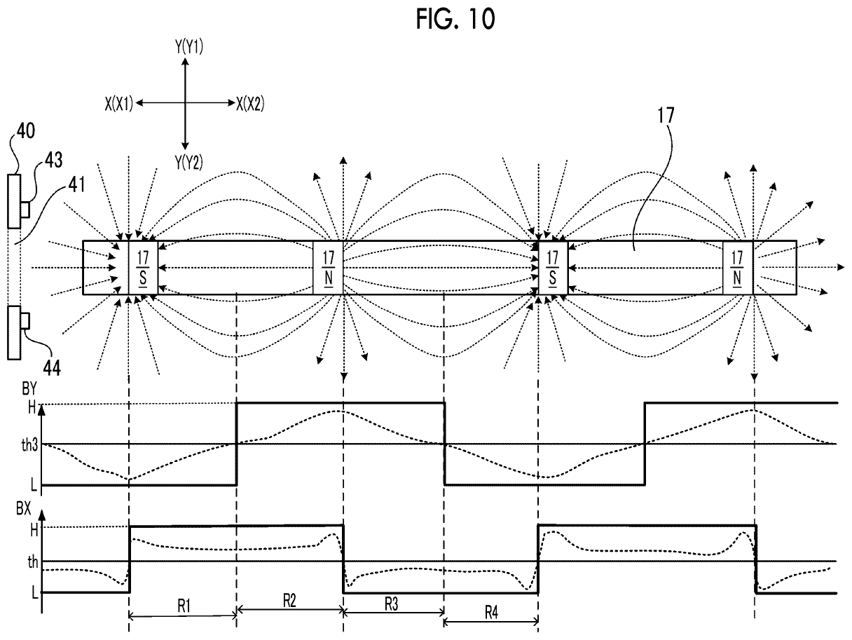

In FIG. 10, the result (classification level) of classifying the magnetic flux density BX and the magnetic flux density BY in the graphs illustrated in FIG. 8 is indicated by a thick solid line. As illustrated in FIG. 10, in the tubular member 17, a range between two adjacent positions P1 is divided into a region R1 in which the magnetic flux density BX is at the high level and the magnetic flux density BY is at the low level, a region R2 in which the magnetic flux density BX is at the high level and the magnetic flux density BY is at the high level, a region R3 in which the magnetic flux density BX is at the low level and the magnetic flux density BY is at the high level, and a region R4 in which the magnetic flux density BX is at the low level and the magnetic flux density BY is at the low level. As described above, the range between the negative pole ends adjacent to each other in the longitudinal direction X can be divided into four regions R1 to R4 depending on the combination of the classification level of the magnetic flux density BX and the classification level of the magnetic flux density BY. By monitoring the thick solid lines (classification levels of the magnetic flux densities BX and BY) illustrated in FIG. 10, the processor 8P can determine the movement direction of the insertion part 10 and the movement amount (movement distance) of the insertion part 10 in the longitudinal direction X.

In the description above, the processor 8P classifies the magnetic flux density into the plurality of pieces of information according to the magnitude thereof. However, a configuration may be adopted in which this classification is performed by a processor provided in the communication chip of the detection unit 40. That is, a configuration may be adopted in which the detection unit 40 transmits information on the classification level indicated by the thick solid line illustrated in FIG. 9 or FIG. 10 to the processor 8P. In addition, the processor 8P performs the determination of the movement state of the insertion part 10, but a configuration may be adopted in which the processor provided in the communication chip of the detection unit 40 performs the determination to transmit the determination result to the processor 8P. Further, a configuration may be adopted in which a processor such as a personal computer connected to the expansion device 8 via a network acquires the information on the magnetic flux density from the detection unit 40, performs the determination, and transmits the determination result to the processor 8P. Also, a processor separate from the processor 8P may perform the determination of the movement state of the insertion part 10. Further, a configuration may be adopted in which a processor provided outside the endoscope device 100 performs the determination of the movement state of the insertion part 10 to transmit the determination result to the processor 8P.

The threshold value used in a case of classifying each of the magnetic flux density BX and the magnetic flux density BY according to the magnitude thereof may be a predetermined fixed value, but the threshold value is preferably a variable value to be determined on the basis of the magnetic flux densities detected by the magnetic detection unit 43 and the magnetic detection unit 44 after the insertion of the insertion part 10 into the through-hole 41 is started.

For example, in a case in which the start-up switch of the detection unit 40 is turned on, the insertion part 10 is inserted into the through-hole 41, and the third magnetic pole region from the most distal end side of the tubular member 17 passes through the through-hole 41, the processor 8P can acquire each of the maximum value and the minimum value of the magnetic flux density BX detected by the magnetic detection unit 43, and the maximum value and the minimum value of the magnetic flux density BY detected by the magnetic detection unit 43. In a case where the maximum value and the minimum value of the magnetic flux density BX are acquired, the processor 8P obtains an average value of the maximum value and the minimum value, and sets the average value as the first threshold value th. Further, in a case in which the maximum value and the minimum value of the magnetic flux density BY are acquired, the processor 8P obtains an average value of the maximum value and the minimum value, sets a value obtained by adding a predetermined value to the average value as the second threshold value th1, and sets a value obtained by subtracting a predetermined value from the average value as the second threshold value th2. The predetermined value is a value that is larger than a value assumed as the disturbance component and is less than the absolute value of each of the maximum value and the minimum value of the magnetic flux density BY. The first to third magnetic pole regions from the most distal end side of the tubular member 17 constitute a base end part on the demagnetized region (adjacent region) side in the region in which the magnetic pattern is formed.

Hereinafter, the processor 8P need only classify the magnetic flux density BX and the magnetic flux density BY by using the threshold values set in this manner. In this way, it is possible to perform the determination of the movement state of the insertion part 10 with higher accuracy by setting the threshold values on the basis of the magnetic flux densities detected by the magnetic detection unit 43 and the magnetic detection unit 44.

In this way, in a case in which the threshold value is set on the basis of the magnetic flux densities detected by the magnetic detection unit 43 and the magnetic detection unit 44, it is preferable that, in a period until the third magnetic pole region from the most distal end side of the tubular member 17 passes through the through-hole 41, the processor 8P sets the first threshold value th, the second threshold value th1, and the second threshold value th2 to the predetermined values, performs the detection of the reference position and the determination of the movement state of the insertion part 10, and then updates the first threshold value th, the second threshold value th1, and the second threshold value th2 by the method described above to perform the determination of the movement state of the insertion part 10.

As described above, in the endoscope system 200, the magnetic pattern is formed on the tubular member 17 such that the magnetic flux densities BX and BY detected by each of the magnetic detection unit 43 and the magnetic detection unit 44 are changed periodically between the positive side and the negative side and phases thereof are shifted in a case in which the insertion part 10 passes through the through-hole 41, so that it is possible to perform the determination of the movement state of the insertion part 10. Such a magnetic pattern is not limited to the configurations of the magnetic pole portions MA1 and MA2 illustrated in FIGS. 3 and 4, and can be variously modified.

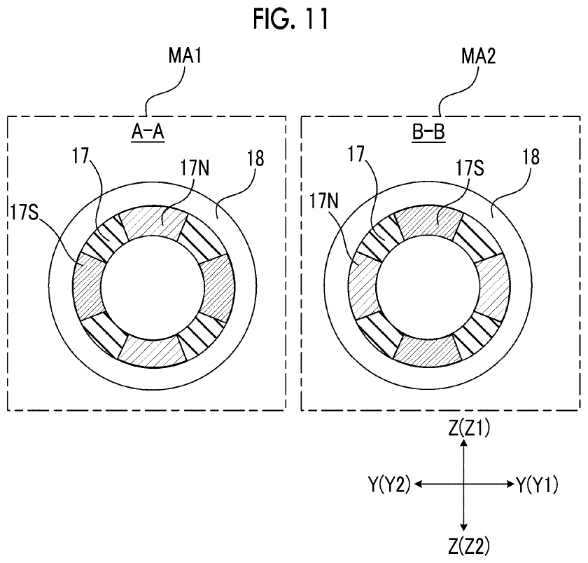

FIG. 11 is a schematic cross-sectional view illustrating a modification example of the magnetic pole portions MA1 and MA2 illustrated in FIG. 3 taken along the A-A arrow and the B-B arrow. In the modification example illustrated in FIG. 11, the magnetic pole portion MA1 has a configuration in which the negative pole region 17S and the positive pole region 17N are formed alternately with an interval therebetween along the circumferential direction of the tubular member 17. Similarly, the magnetic pole portion MA2 has a configuration in which the negative pole region 17S and the positive pole region 17N are formed alternately with an interval therebetween along the circumferential direction of the tubular member 17. The magnetic pole portion MA2 has a configuration in which the magnetic pole portion MA1 is rotated by 90 degrees around an axis center of the tubular member 17.

As illustrated in FIG. 11, in a state of being viewed in the longitudinal direction X, the positive pole region 17N in the magnetic pole portion MA1 and the negative pole region 17S in the magnetic pole portion MA2 are present at the same position in the circumferential direction of the tubular member 17. That is, in the tubular member 17, all the magnetic pole regions at the same position in the circumferential direction have a configuration in which the negative pole region 17S and the positive pole region 17N are alternately arranged in the longitudinal direction X. That is, in the tubular member 17, a first magnetic pattern in which the negative pole region 17S and the positive pole region 17N are alternately arranged along the longitudinal direction X with the negative pole region 17S at the beginning, and a second magnetic pattern in which the negative pole region 17S and the positive pole region 17N are alternately arranged along the longitudinal direction X with the positive pole region 17N at the beginning are alternately arranged with an interval therebetween in the circumferential direction of the tubular member 17.

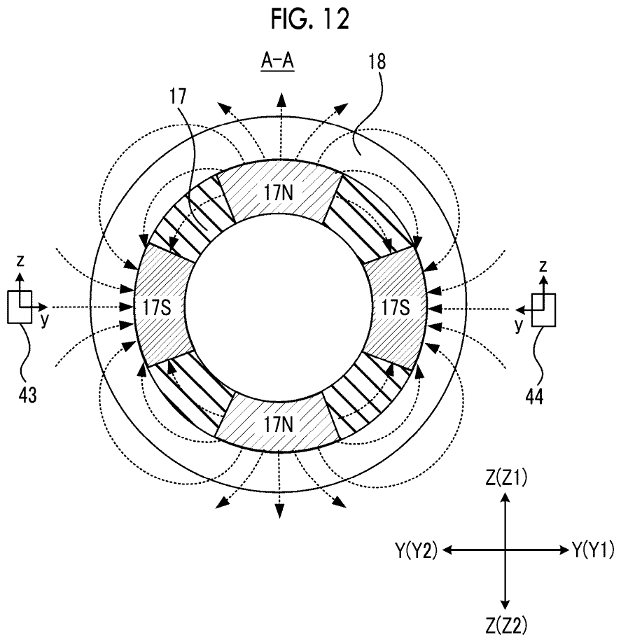

FIG. 12 is a diagram schematically illustrating a magnetic flux line generated in the magnetic pole portion MA1 having the configuration illustrated in FIG. 11. FIG. 12 illustrates the positions of the magnetic detection units 43 and 44 with respect to the soft portion 10A in a case in which the soft portion 10A passes through the through-hole 41.

In a state illustrated in FIG. 12, the magnetic flux density BY detected by the magnetic detection unit 43 has a large negative value. In a case in which the soft portion 10A is rotated by 45 degrees counterclockwise from the state illustrated in FIG. 12, the magnetic flux density BY detected by the magnetic detection unit 43 has a value close to zero. In a case in which the soft portion 10A is rotated by 90 degrees counterclockwise from the state illustrated in FIG. 12, the magnetic flux density BY detected by the magnetic detection unit 43 has a large positive value. In a case in which the soft portion 10A is rotated by 135 degrees counterclockwise from the state illustrated in FIG. 12, the magnetic flux density BY detected by the magnetic detection unit 43 has a value close to zero. In a case in which the soft portion 10A is rotated by 180 degrees counterclockwise from the state illustrated in FIG. 12, the magnetic flux density BY detected by the magnetic detection unit 43 has a large negative value. In a case in which the soft portion 10A is rotated in the circumferential direction thereof in the through-hole 41 in this manner, the magnetic flux density BY detected by the magnetic detection unit 43 is equivalent to the magnetic flux density BY illustrated in FIG. 8. Similarly, in a case in which the soft portion 10A is rotated in the circumferential direction thereof in the through-hole 41, the magnetic flux density BZ detected by the magnetic detection unit 43 is equivalent to the magnetic flux density BY illustrated in FIG. 8 and has a phase shifted by 90 degrees. Therefore, in a case in which the magnetic flux densities BY and BZ detected by the magnetic detection unit 43 are classified into the high level and the low level, respectively, these classification levels are equivalent to the thick solid lines of the magnetic flux density BY illustrated in FIG. 10 (it should be noted that the phases of the magnetic flux density BY and the magnetic flux density BZ are shifted by 90 degrees). Therefore, it is possible to derive a rotation direction and a rotation amount of the insertion part 10 by the combination of the classification levels.

In a case in which the endoscope 1 having the magnetic pattern having such a configuration is used, the processor 8P can determine a rotation state (rotation direction and rotation amount (rotation angle)) of the insertion part 10 in the circumferential direction in the same manner as the determination method of the movement state of the insertion part 10, by classifying each of the magnetic flux density BZ and the magnetic flux density BY into the plurality of pieces of information and monitoring the change in the combination of these pieces of information. In the configuration illustrated in FIG. 11, since the first magnetic pattern and the second magnetic pattern extending in the longitudinal direction X are formed on the tubular member 17, it is possible to determine the movement state of the insertion part 10 on the basis of the magnetic flux density BX and the magnetic flux density BY, as described above. In FIG. 11, each of the magnetic pole portions MA1 and the magnetic pole portions MA2 includes four magnetic pole regions arranged in the circumferential direction. However, each of the magnetic pole portion MA1 and the magnetic pole portion MA2 may have a configuration of including two magnetic pole regions, or have a configuration of including an even number (six or more) of magnetic pole regions.

Even in the configuration illustrated in FIG. 11, it is preferable that the arithmetic means of the magnetic flux densities BY and BZ detected by the magnetic detection unit 43 and the magnetic flux densities BY and BZ detected by the magnetic detection unit 44 are obtained, each of the values of these two arithmetic means is classified into the high level and the low level, and the rotation direction and the rotation amount of the insertion part 10 are derived by the combination of the classification levels.

### <Processing of Processor 8P>

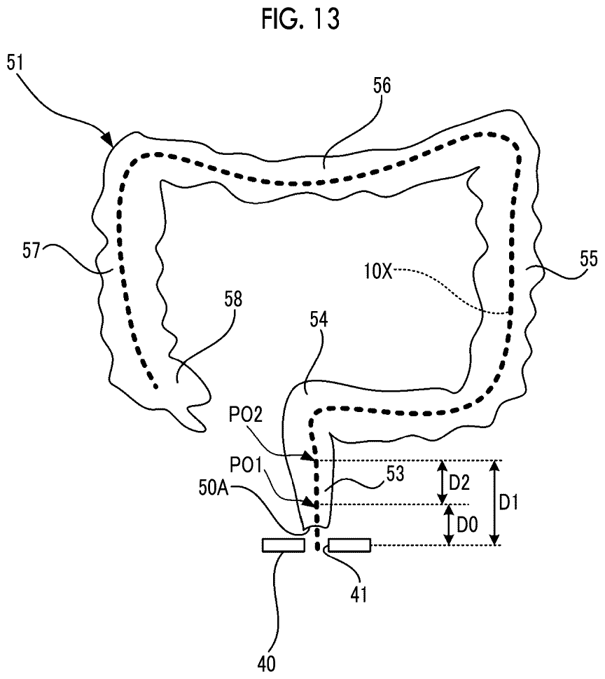

Next, details of various kinds of processing executed by the processor 8P will be described. In order to describe these various kinds of processing, the movement path of the insertion part 10 of the endoscope 1 will be described. FIG. 13 is a schematic diagram illustrating the movement path of the insertion part 10 in an examination (hereinafter, referred to as endoscopy) performed using the endoscope 1.

The endoscopy includes an endoscopy that examines an upper digestive organ such as a stomach, an endoscopy that examines a lower digestive organ such as a large intestine. In addition, the endoscopy includes a first examination in which the insertion part 10 is inserted into the subject in order to examine whether or not a lesion region is present in the subject, and a second examination in which the insertion part 10 is inserted into the subject in order to excise the already known lesion region.

### (Movement Path of Endoscope)

FIG. 13 illustrates a large intestine 51 of the subject (subject 50). In the endoscopy of the large intestine, the insertion part 10 is moved along a movement path 10X indicated by the broken line in the drawing. The movement path 10X is a tubular path from the through-hole 41 of the detection unit 40 disposed in the vicinity of the anus 50A outside the subject through the anus 50A to a rectum 53, and further from the rectum 53 through a sigmoid colon 54, a descending colon 55, a transverse colon 56, and an ascending colon 57 to an ileocecum 58.

In the endoscopy and the first examination of the large intestine, the operator of the endoscope 1 inserts the insertion part 10 into the anus 50A through the through-hole 41, causes the insertion part 10 to reach the ileocecum 58 which is a turnaround point of the examination, and then pulls out the insertion part 10 from the ileocecum 58 toward the outside of the subject. Hereinafter, a step of moving the distal end of the insertion part 10 from the through-hole 41 to the ileocecum 58 will be described as an insertion step, and a step of moving the distal end of the insertion part 10 from the ileocecum 58 to the through-hole 41 will be referred to as a pulling-out step. The first examination is composed of a set of the insertion step and the pulling-out step. The endoscopy and the second examination of the large intestine are the same as the first examination except that the turnaround point of the examination is changed to the presence position of the lesion region found in the first examination in advance.

In the endoscopy of the stomach, the turnaround point of the first examination is the duodenum, and the turnaround point of the second examination is the presence position of the lesion region found in the first examination in advance.

### (Processing During Endoscopy)

In a case where the endoscopy is started, the power of the detection unit 40 is turned on. As described above, the processor 8P derives a first distance (the insertion length described above) from the reference position (position of the through-hole 41) on the movement path 10X to the distal end of the insertion part 10 on the basis of the magnetic flux densities BX and BY detected by the detection unit 40.

### (Determination of Reaching Site)

In a case in which the endoscope 1 is activated, the processor 8P performs reaching site determination processing of sequentially acquiring the captured images captured by the endoscope 1, and determining the site (the anus, the rectum, the sigmoid colon, the top part of the sigmoid colon (S-top), the transition part between the sigmoid colon and the descending colon (SDJ), the descending colon, the splenic flexure, the transverse colon, the hepatic curvature, the ascending colon, the ileocecum, or the outside of the body and the like) in the subject that the distal end of the insertion part 10 has reached on the basis of the acquired captured images and the derived first distance. The processor 8P performs the reaching site determination processing using, for example, a recognition model (machine learning model) generated by machine learning, and the first distance.

### (First Determination Example of Reaching Site)

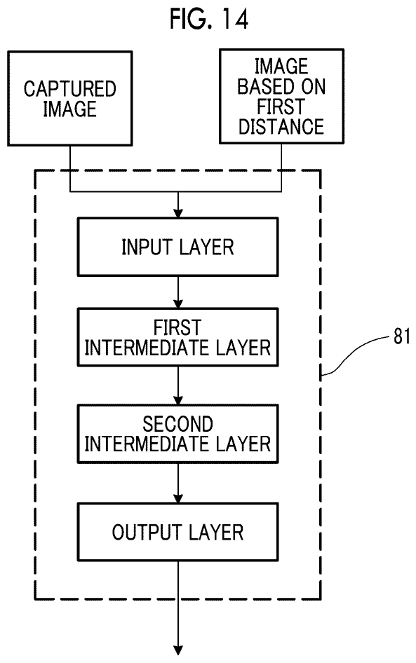

FIG. 14 is a schematic diagram for describing a first determination example of a reaching site. FIG. 14 illustrates a recognition model 81. The recognition model 81 comprises an input layer, at least one intermediate layer (two intermediate layers, a first intermediate layer and a second intermediate layer in the example illustrated in FIG. 14), and an output layer, and a fully connected layer that connects these layers. The recognition model 81 is generated by being trained, for example, using a set of a captured image of a specific site acquired in a past endoscopy and an image based on a first distance in a case of reaching the specific site acquired in the past endoscopy (hereinafter, also referred to as a distance image) as training data, to output answer data indicating that the reaching site is the specific site. The combination of the training data and the answer data is prepared for each site in the subject, and learning is performed for each site.

As the first distance that is a source of the distance image used for the training data, a value measured by the endoscope device 100 (for example, a measured value of the first distance in a case where the operator determines that the endoscope device 100 has reached a specific site) may be used, or a value statistically determined from anatomical knowledge (for example, information indicating that the ileocecum is at a distance of several cm from the position of the detection unit 40) may be used. The distance image is, for example, an image obtained by converting the first distance into an image of characters or the like, or an image obtained by converting a reaching site in the subject, which is statistically determined from the first distance, into an image of characters or the like.

In a case where the endoscope 1 is activated, the processor 8P sequentially acquires the captured image obtained by imaging with the endoscope 1, and inputs the acquired captured image and the image based on the derived first distance to the recognition model 81. The recognition model 81 that has received this input outputs a recognition result (the recognition site and the correct answer rate thereof) of the reaching site. In a case where the correct answer rate is equal to or greater than the threshold value, the processor 8P determines that the site in the subject reached by the distal end of the insertion part 10 is the recognition site included in the recognition result.

### (Second Determination Example of Reaching Site)

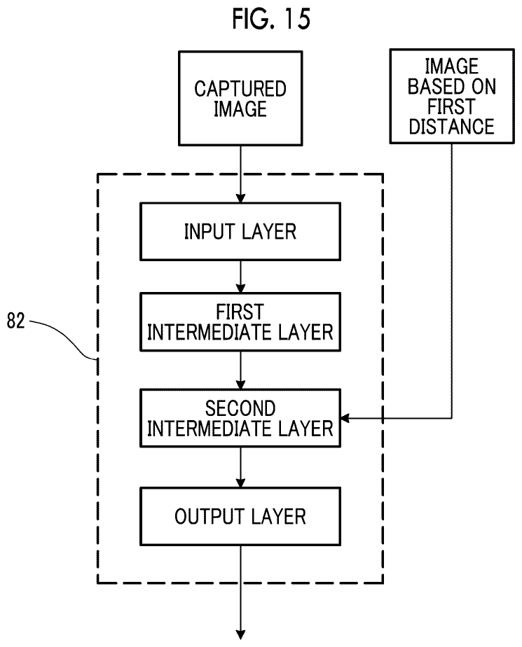

FIG. 15 is a schematic diagram for describing a second determination example of a reaching site. The recognition model 82 illustrated in FIG. 15 is generated by learning a combination of the training data and the answer data in the same manner as the recognition model 81, but is different from the recognition model 81 in that an input destination of the distance image as the training data is the second intermediate layer instead of the input layer. In the recognition model 82, the first intermediate layer extracts, for example, a feature amount from the captured image of the training data. The recognition model 82 is trained by the feature amount and the distance image being input to the second intermediate layer as training data. The combination of the training data and the answer data is prepared for each site in the subject, and learning is performed for each site.

In a case where the endoscope 1 is activated, the processor 8P sequentially acquires the captured image obtained by imaging with the endoscope 1, inputs the acquired captured image to the input layer of the recognition model 82, and inputs the image based on the derived first distance to the second intermediate layer of the recognition model 82. The recognition model 82 that has received this input outputs a recognition result (the recognition site and the correct answer rate thereof) of the reaching site. In a case where the correct answer rate is equal to or greater than the threshold value, the processor 8P determines that the site in the subject reached by the distal end of the insertion part 10 is the recognition site included in the recognition result.

### (Third Determination Example of Reaching Site)

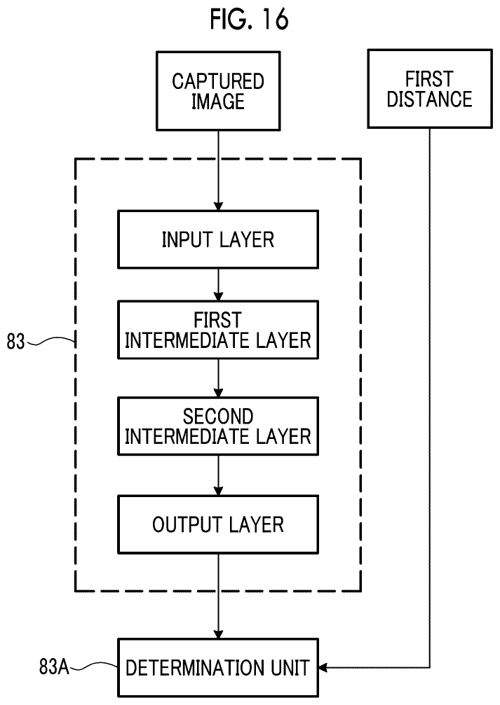

FIG. 16 is a schematic diagram for describing a third determination example of a reaching site. The recognition model 83 illustrated in the FIG. 16 comprises an input layer, at least one intermediate layer (two intermediate layers, a first intermediate layer and a second intermediate layer in the example illustrated in FIG. 16), and an output layer, and a fully connected layer that connects these layers. The recognition model 83 is generated by being trained, for example, using a captured image of a specific site acquired in a past endoscopy as training data, to output answer data indicating that the reaching site is the specific site. The combination of the training data and the answer data is prepared for each site in the subject, and learning is performed for each site.

The determination unit 83A illustrated in FIG. 16 is a functional block of the processor 8P. The determination unit 83A acquires the recognition result (the recognition site and the correct answer rate thereof) by the recognition model 83, and determines where the reaching site is based on the recognition result and the first distance derived in a state in which the recognition result is obtained. For example, in a case where information on a reaching site corresponding to the first distance is acquired from table data in which a correspondence relationship between the first distance and the reaching site is statistically obtained, and the information and the recognition site included in the recognition result match and the correct answer rate included in the recognition result is equal to or higher than a threshold value, it is determined that the site in the subject reached by the distal end of the insertion part 10 is the recognition site included in the recognition result.

The processor 8P may perform the reaching site determination processing only in a case where a predetermined condition is satisfied, instead of sequentially performing the reaching site determination processing after the endoscope 1 is activated. The predetermined condition is, for example, that a specific recognition result is obtained by the recognition processing related to the endoscopy (the lesion recognition processing, the treatment tool recognition processing, or the like described above), that a recording instruction of the captured image is given, and the like.

For example, in a case where the lesion region is detected based on the captured image, the processor 8P performs the above-described reaching site determination processing based on the captured image and the first distance derived at that time to determine where the site in the subject in which the lesion region is detected is. In addition, for example, in a case where the treatment tool is detected based on the captured image, the processor 8P performs the above-described reaching site determination processing based on the captured image and the first distance derived at that time to determine which site in the subject has been treated. In this case, it is preferable that the processor 8P stores the result of the lesion recognition processing or the treatment tool recognition processing (the result of detecting the lesion region or the result of performing the treatment), the reaching site determined by the reaching site determination processing, and the first distance used in the reaching site determination processing in the memory in association with each other. In this way, it is possible to check the position of the lesion region or the position where the treatment is performed after the examination.

### (Modification Example of Recognition Model)

The training data used for each of the generation of the recognition model 81 and the recognition model 82 may be a set of a plurality of captured images (a plurality of captured images arranged in time series) continuously obtained for a predetermined period of time in a case where a specific site is reached in a past endoscopy, and an image based on each of a plurality of first distances (a plurality of first distances arranged in time series) continuously derived for a predetermined period of time in a case where the specific site is reached, instead of a set of single captured image and single distance image. The training data used for generating the recognition model 83 may be a plurality of captured images continuously obtained for a predetermined period of time (a plurality of captured images arranged in time series) instead of a single captured image. In such a case, in the example of FIGS. 14 and 15, the processor 8P may input, for example, after the endoscope 1 is activated, the captured image acquired at the first timing, the captured image acquired at the second timing after the first timing, the image based on the first distance derived at the first timing, and the image based on the first distance acquired at the second timing, to the recognition model, and may determine the reaching site based on the output of the recognition model.

The training data used for generating each of the recognition model 81 and the recognition model 82 may further include a change amount per unit time of the first distance (in other words, the movement speed of the endoscope 1).

For example, the recognition model 81 may be generated by being trained using a set of the captured image of the specific site acquired in the past endoscopy, the image based on the first distance in a case of reaching the specific site acquired in the past endoscopy, and the change amount per unit time of the first distance derived in a case of reaching the specific site in the past endoscopy as training data, to output the answer data indicating that the reaching site is the specific site. In such a case, for example, after the endoscope 1 is activated, the processor 8P may input the captured image acquired at the first timing, the captured image acquired at the second timing after the first timing, the image based on the first distance derived at the first timing, the image based on the first distance acquired at the second timing, and the change amount in the first distance in the time from the second timing to the first timing, to the recognition model 81, and determine the reaching site based on the output of the recognition model.

In the insertion step, the movement speed of the endoscope 1 may greatly change depending on the reaching site. By learning this movement speed and recognizing the reaching site, it is possible to improve the recognition accuracy. In a case where the distal end of the endoscope 1 has reached the ileocecum, the endoscope 1 is sufficiently inserted to the inside, and thus the movement speed of the endoscope 1 tends to decrease. Therefore, by considering the movement speed, it is possible to recognize that the reaching site is the ileocecum with high accuracy. For example, in a case where it is estimated that the reaching site is the ileocecum from the captured image, it is estimated that the reaching site is the ileocecum from the first distance, and it is further estimated that the reaching site is the vicinity of the ileocecum from the movement speed, the determination result that the reaching site is the ileocecum can be output.

The processor 8P can also determine, for example, whether any of the insertion step or the pulling-out step is performed by using the result of the reaching site determination processing. As an example, the processor 8P determines a period after the determination result that the reaching site is the anus 50A or the rectum 53 is obtained until the determination result that the reaching site is the ileocecum 58 is obtained, as a period (first period) of the insertion step in which the endoscope 1 is moved from a starting point toward an ending point of the movement path 10X, and determines a period after the determination result that the reaching site is the ileocecum 58 is obtained until the determination result that the reaching site is the outside of the subject is obtained, as a period (second period) of the pulling-out step in which the endoscope 1 is moved from the ending point toward the starting point of the movement path lox.

The processor 8P can determine the movement direction of the insertion part 10 on the movement path 10X on the basis of a time change of the first distance derived on the basis of the magnetic flux densities BX and BY detected by the detection unit 40, and can discriminate the period of the insertion step and the period of the pulling-out step from the movement direction. For example, in a case where the first distance tends to be increased, the processor 8P determines that the insertion part 10 is being moved in a direction from the outside of the body of the subject toward the ileocecum 58, and determines the period of the insertion step (first period). On the other hand, in a case where the first distance tends to be decreased, the processor 8P determines that the insertion part 10 is being moved from the ileocecum 58 toward the outside of the body of the subject, and determines the period of the pulling-out step (second period).

The recognition model 83 described above is generated by machine learning, but a method of recognizing a site by general image processing may be employed.

### (Detection of Event)

The processor 8P can detect the occurrence of various events related to the endoscopy by using, for example, the result of the above-described reaching site determination processing and the result of the above-described lesion recognition processing and treatment tool recognition processing to acquire event information which is information on the event.

For example, the processor 8P can detect an event that the insertion step is started, an event that the pulling-out step is started, an event that the endoscopy is ended, an event that the distal end of the endoscope 1 reaches a specific site in the subject, an event that a specific operation (for example, operation of the treatment tool) of the endoscope 1 is performed, an event that the lesion region is detected from the subject, or the like.

Specifically, in a case where the determination result that the reaching site is the anus 50A is obtained by the reaching site determination processing, the processor 8P detects the occurrence of the event that the endoscopy is started (insertion step is started) (examination start event). In a case where, after the examination start event is detected, the recognition result that the reaching site is the ileocecum 58 is obtained by the reaching site determination processing, the processor 8P detects the occurrence of the event that the pulling-out step is started (pulling-out start event). In a case where, after the pulling-out start event, the recognition result that the reaching site is not inside the subject is obtained, the processor 8P detects the occurrence of the event that the endoscopy is ended (examination end event).

In a case where the lesion region is detected by the lesion recognition processing, the processor 8P detects the occurrence of the event that the lesion region is detected (lesion detection event). In a case where the treatment tool is detected by the treatment tool recognition processing, the processor 8P detects the occurrence of the event that the treatment (operation of the treatment tool) is performed (treatment event). In a case where the determination result that a predetermined specific site is reached is obtained by the reaching site determination processing, the processor 8P detects the event that the distal end of the insertion part 10 reaches the specific site (specific site reaching event).

### (Derivation of Second Distance)

The processor 8P may derive a second distance, which is a distance of the distal end of the insertion part 10 from the predetermined site in the subject, based on the result of the above-described reaching site determination processing and the first distance derived based on the magnetic flux densities BX and BY.

First, in a case where the endoscopy of the large intestine is started, in the initial stage of the insertion step, the processor 8P obtains the determination result that the reaching site of the distal end of the insertion part 10 is the anus 50A or the rectum 53. In a case where such a determination result is obtained, the processor 8P sets the first distance derived in a state in which the determination result is obtained as the first correction value. Then, after the determination result is obtained, the processor 8P performs processing of subtracting the first correction value from the first distance derived based on the magnetic flux densities BX and BY to obtain the specific insertion length (a distance from the reference position to the distal end of the insertion part 10 in a case where the anus 50A or the rectum 53 on the starting point side of the movement path 10X is used as the reference position). By this processing, in the insertion step, the second distance in a case where the anus 50A or the rectum 53 is the predetermined site (first predetermined site) is sequentially derived as the specific insertion length. For example, as illustrated in FIG. 13, a case is assumed in which the determination result that the reaching site is the rectum 53 in a state where the distal end of the insertion part 10 reaches a position PO1 is obtained. In this case, in a case where the distal end of the insertion part 10 slightly advances from the rectum 53 to be moved to a position PO2, a value (=D2) obtained by subtracting the first distance (=D0, first correction value) derived in a state where the distal end of the insertion part 10 is at the position PO1, from the first distance (=D1) derived at a point in time when the distal end of the insertion part 10 is moved to the position PO2, is derived as the specific insertion length.

After that, in a case where the insertion step is continued and the distal end of the insertion part 10 is moved to a turnaround point (that is, the ileocecum 58) at which the insertion step is switched to the pulling-out step, the processor 8P obtains the determination result that the reaching site of the distal end of the insertion part 10 is the ileocecum 58. In a case where the determination result that the reaching site is the ileocecum 58 is obtained, the processor 8P sets the first distance derived in a state in which the determination result is obtained, as the second correction value. Then, after the determination result is obtained, the processor 8P performs processing of obtaining the pulling-out length (a distance from the reference position to the distal end of the insertion part 10 in a case where the ileocecum 58 at the ending point of the movement path 10X is set as the reference position) by subtracting the first distance derived based on the magnetic flux densities BX and BY from the second correction value. By this processing, in the pulling-out step, the second distance in a case where the ileocecum 58 is the predetermined site (second predetermined site) is sequentially derived as the pulling-out length.

In the insertion step of the endoscopy of the large intestine, the insertion part 10 may be inserted while the large intestine is folded, or the insertion part 10 may be inserted while the large intestine is stretched. On the other hand, in the pulling-out step of the endoscopy of the large intestine, the insertion part 10 is pulled out in a state where the large intestine has returned to a steady state. Therefore, in the endoscopy of the large intestine, even in a case where the first distances derived on the basis of the magnetic flux densities BX and BY are the same in the insertion step and the pulling-out step, the positions at which the distal end of the insertion part 10 is present in the large intestine 51 are different in some cases. In the present embodiment, in the insertion step, the front end position of the insertion part 10 can be managed by the specific insertion length, and in the pulling-out step, the front end position of the insertion part 10 can be managed by the pulling-out length. Therefore, the insertion state of the insertion part 10 can be managed with high accuracy.

The specific insertion length constitutes a distance from the reference position (position of the anus 50A or the rectum 53) on the starting point side of the movement path 10X to the distal end of the endoscope 1 moved along the movement path 10X. The pulling-out length constitutes a distance from the ending point position (the position of the ileocecum 58) on the movement path 10X to the distal end of the endoscope 1 moved along the movement path 10X.

The first distance constitutes a distance from the reference position (position of the through-hole 41) on the starting point side of the movement path 10X to the distal end of the endoscope 1 moved along the movement path 10X. The first distance, the specific insertion length, or the pulling-out length will also be referred to distance information below.

The recognition model 81 illustrated in FIG. 14 is generated by training the first distance as training data. The specific insertion length or pulling-out length may be used instead of the first distance as the training data for generating the recognition model 81. A recognition model generated using the specific insertion length instead of the first distance as the training data for generating the recognition model 81 will be referred to as a recognition model 81A below. A recognition model generated using the pulling-out length instead of the first distance as the training data for generating the recognition model 81 will be referred to as a recognition model 81B below.

In a case where the endoscope 1 is activated, the processor 8P first determines the reaching site of the distal end of the endoscope 1 by using the recognition model 81, the captured image, and the first distance. In a case where it is determined that the reaching site is the anus or the rectum, the processor 8P determines the reaching site of the distal end of the endoscope 1 using the recognition model 81A, the captured image, and the specific insertion length. Thereafter, in a case where the processor 8P determines that the reaching site is the ileocecum, the processor 8P determines the reaching site of the distal end of the endoscope 1 using the recognition model 81B, the captured image, and the pulling-out length. As described above, by performing the determination of the reaching portion using different recognition models in the insertion step and the pulling-out step, it is possible to improve the determination accuracy of the reaching site in the insertion step and the determination accuracy of the reaching site in the removal step.

### (Display and Recording During Endoscopy)

In the period of the insertion step, it is preferable that the processor 8P performs control to display at least one of the specific insertion length (second distance) or the first distance derived as described above on the display device 7, or performs control to record the specific insertion length or the first distance in association with the information regarding the endoscopy (hereinafter, referred to as examination association information) in the recording medium (for example, the memory of the expansion device 8). The examination association information refers to the captured image captured by the endoscope 1, various kinds of event information described above, an elapsed time (examination time) from the start of the endoscopy (examination start event), and the like. For example, each time the first distance and the specific insertion length are derived, the processor 8P performs control to record which derived value is associated with the elapsed time (examination time). In a case where there is an instruction to record the captured image, the processor 8P performs control to record the captured image further in association with the elapsed time at that time. In a case where the event information is acquired, the processor 8P performs control to record the event information in association with the elapsed time at that time.

In the period of the pulling-out step, it is preferable that the processor 8P performs control to display at least one of the pulling-out length (the second distance) or the first distance derived as described above on the display device 7, or performs control to record the pulling-out length or the first distance in association with the examination association information in the recording medium.

The processor 8P may perform control of outputting the operation support information based on the reaching site determined by the reaching site determination processing. For example, in the insertion step, depending on the position of the distal end of the insertion part 10, the hardness adjustment of the insertion part 10 of the endoscope 1 or the manual compression may be required in order to smoothly insert the insertion part 10. For example, in a case where it is determined that the reaching site is a site where the hardness adjustment or manual compression is required, the processor 8P performs control of displaying information (operation support information) for instructing the hardness adjustment or manual compression on the display device 7, or performs control of outputting the information by voice from the speaker. In this manner, it is possible to smoothly insert the endoscope 1. The processor 8P may perform control of outputting the operation support information only in the insertion step of the insertion step and the removal step based on the result of the reaching site determination processing, and may not perform the control in the pulling-out step. In the pulling-out step of the endoscopy of the large intestine, it is often not difficult to pull out the endoscope 1, and therefore, it is possible to reduce the processing load of the processor 8P by doing in this manner.

The examination data including the examination association information (the captured image, the event information, or the examination time) and the distance information (the first distance, the specific insertion length, or the pulling-out length) associated by the processor 8P is transmitted to a server (not illustrated) and stored. After the endoscopy is ended, an examination report creation support device that can access the server creates a draft of an examination report on the basis of the examination data. A doctor can efficiently perform work by creating a final examination report using the draft.

### (Display Example of Examination Data)

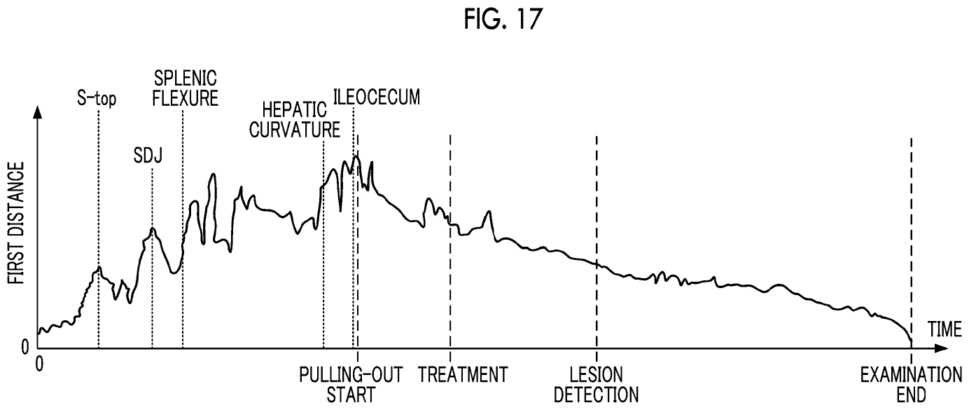

FIG. 17 is a graph illustrating a display example of the examination data associated and recorded by the processor 8P. The processor 8P performs control to display the graph illustrated in FIG. 17 on, for example, the display device 7 or another display. With the graph displayed in this way, the operator of the endoscope 1 or an instructor thereof can evaluate the procedure of the endoscopy.

In the graph illustrated in FIG. 17, the first distance is plotted for each elapsed time of the endoscopy. In the graph illustrated in FIG. 17, characters (S-top, SDJ, splenic flexure, hepatic curvature, and ileocecum) indicating the content (reaching site) of the specific site reaching event are attached to the timing when the specific site reaching event is detected. In addition, characters (pulling-out start, treatment, lesion detection, examination end) indicating the content of another event are attached to the timing at which the event is detected. The period from the start of the insertion step (elapsed time=0) to the pulling-out start event is the period of the insertion step, and the period from the pulling-out start event to the examination end event is the period of the pulling-out step.

In a case where an arbitrary position in the plot waveform in FIG. 17 is designated and a captured image associated with the elapsed time of the arbitrary position is recorded, the processor 8P may display the captured image on the display device 7.

### <Main Effects of Endoscope System 200>

According to the endoscope system 200, since the reaching site of the distal end of the endoscope 1 is determined based on the captured image and the distance information, it is possible to improve the determination accuracy.

With the endoscope system 200, not only the insertion length (first distance) of the insertion part 10 into the subject with the position of the detection unit 40 installed outside the subject as the starting point, but also the specific insertion length of the insertion part 10 into the subject with the first predetermined site (anus or rectum) in the subject as the starting point and the pulling-out length of the insertion part 10 to the outside of the subject with the second predetermined site (ileocecum) in the subject as the starting point can be derived. In a case of performing the endoscopy of the stomach, it is possible to obtain the specific insertion length and the pulling-out length by setting the first predetermined site as, for example, a cardia, and the second predetermined site as, for example, a duodenum.

With the endoscope system 200, since the specific insertion length and the pulling-out length are derived by using the result of the reaching site recognition processing using the captured image obtained through the imaging by the endoscope 1 actually inserted into the subject, it is possible to eliminate the influence of individual differences for each subject, and manage the front end position of the insertion part 10 with high accuracy by using the specific insertion length and the pulling-out length. As a result, it is possible to perform the operation support of the endoscope 1 with high accuracy during the endoscopy. In addition, it is possible to determine the recording position of the captured image with high accuracy, which can be used for later creation of an examination report or can improve the diagnosis accuracy. In particular, since the specific insertion length and the pulling-out length can be derived separately, these effects can be further enhanced.

As described above, the detection unit 40 can also be integrally configured with an insertion assisting member of the endoscope 1. For example, the detection unit 40 may be integrally formed with the insertion assisting member to be inserted into the anus, or may be integrally formed with a mouthpiece-type insertion assisting member that is held in the mouth. In addition, the detection unit 40 may be integrally formed with the pants for endoscopy, or may be configured to be attachable to and detachable from the pants for endoscopy.

The technology of the present disclosure is not limited to the above description, and can be appropriately changed as described below.

For example, the endoscope 1 may be inserted into the body through the mouth or the nose of the subject 50. In this case, the detection unit 40 need only have a shape to be attachable to the mouth or the nose of the subject 50.

The tubular member 17 has the configuration in which the first member 14 and the second member 15 are provided, and each of the first member 14 and the second member 15 contains the magnetizable austenitic stainless steel, but one of the first member 14 or the second member 15 may be made of a non-magnetizable material. That is, the magnetic pattern may not be formed on one of the first member 14 or the second member 15. Even in such a case, since the magnetic flux densities BX, BY, and BZ described above can be detected from the tubular member 17, it is possible to determine the movement state and the rotation state of the insertion part 10.

In the above description, in the tubular member 17, the two types of magnetic pole regions are alternately arranged in the longitudinal direction to form the magnetic pattern, and the movement state of the insertion part 10 in the longitudinal direction is determined on the basis of the combination of the classification levels of the magnetic information in the two directions detected from the magnetic pattern. However, the two types of magnetic pole regions formed on the tubular member 17 may not be alternately arranged in the longitudinal direction. Even in such a case, the movement state of the insertion part 10 in the longitudinal direction can be determined on the basis of the combination of the classification levels of the magnetic information in the two directions detected from the magnetic pattern.

In addition, as a modification example, the movement state of the insertion part 10 in the longitudinal direction may be determined by forming a pattern more complicated than the magnetic pattern described above on the tubular member 17 and detecting the pattern by the magnetic detection units 43 and 44. Specifically, a table in which each position of the tubular member 17 in the longitudinal direction and the magnetic flux density BX or the magnetic flux density BY (classification level) detected at each position are associated with each other may be recorded in a memory, and the processor 8P may classify the magnetic flux density BX or the magnetic flux density BY detected by the magnetic detection unit 43 to acquire the classification level, and may acquire the information on the position corresponding to the classification level from the table to determine the insertion length of the insertion part 10. As a result, the insertion length of the insertion part 10 can be finely determined. In addition, the magnetic detection units 43 and 44 can detect the magnetic flux densities in one direction, so that the cost can be reduced.

As described above, at least the following matters are described in the present specification.

(1)

A processing device comprising:
a processor configured to:
   acquire a distance from a reference position on a movement path of an endoscope to a distal end of the endoscope that is moved along the movement path;
   acquire a captured image that is captured by the endoscope; and
   determine a reaching site of the distal end of the endoscope inserted into a subject, based on the captured image and the distance.
      (2)

The processing device according to (1),
wherein the processor is configured to determine the reaching site further based on a change amount in the distance per unit time.
   (3)

The processing device according to (2),
wherein the processor is configured to determine whether or not the reaching site is a turnaround point of the distal end of the endoscope in an examination using the endoscope, based on the captured image, the distance in a case where the reference position is a position on the movement path on a starting point side, and the change amount.
   (4)

The processing device according to (3),
wherein the turnaround point includes an ileocecum.
   (5)

The processing device according to any of (1) to (4),
wherein the processor is configured to acquire the distance using different reference positions in a first period in which the endoscope is moving from a starting point to an ending point of the movement path and in a second period in which the endoscope is moving from the ending point to the starting point of the movement path.
   (6)

The processing device according to (5),
wherein the reference position used in the second period is an ending point position of the movement path, and
the reference position used in the first period is a position on a starting point side of the movement path.
   (7)
   The processing device according to (5) or (6),
wherein the processor is configured to determine the reaching site with different processing contents between the first period and the second period.
   (8)

The processing device according to any of (1) to (7),
wherein the processor is configured to:
   perform recognition processing related to an examination of the endoscope based on the captured image; and
   perform determination of the reaching site based on the captured image used in the recognition processing and the distance in a case where a specific recognition result is obtained by the recognition processing.
      (9)

The processing device according to (8),
wherein the processor is configured to store the specific recognition result, a determination result of the reaching site, and the distance in association with one another.
   (10)

The processing device according to any of (1) to (9),
wherein the processor is configured to determine the reaching site based on an output of a machine learning model obtained by inputting an image based on the distance and the captured image to the machine learning model.
   (11)

The processing device according to any of (1) to (9),
wherein the processor is configured to determine the reaching site based on an output of a machine learning model obtained by inputting the captured image to the machine learning model and inputting an image based on the distance to an intermediate layer of the machine learning model.
   (12)

The processing device according to any of (1) to (9),
wherein the processor is configured to determine the reaching site based on an output of a machine learning model obtained by inputting the captured image to the machine learning model, and the distance.
   (13)

The processing device according to any of (1) to (12),
wherein the processor is configured to store information related to an examination of the subject performed using the endoscope and a determination result of the reaching site in association with each other.
   (14)

The processing device according to any of (1) to (13),
wherein the processor in configured to output operation support information of the endoscope based on a determination result of the reaching site.
   (15)

The processing device according to any of (1) to (14),
wherein a magnetic pattern is formed along a longitudinal direction on an insertion part of the endoscope, and
the processor acquires the distance based on a magnetic field from the magnetic pattern, which is detected by a magnetic detection unit installed outside a subject.
   (16)

An endoscope device comprising: the processing device according to any of (1) to (15); and the endoscope.

(17)

The endoscope device according to (16), further comprising:
a magnetic detection unit disposed on the movement path,
wherein an insertion part of the endoscope has a member containing metal, which extends in a longitudinal direction and has a magnetic pattern integrally formed along the longitudinal direction,
the magnetic detection unit detects a magnetic field from the member, and
the processor derives the distance based on the magnetic field detected by the magnetic detection unit.
   (18)

The endoscope device according to (17),
wherein the insertion part includes a soft portion of the endoscope.
   (19)

The endoscope device according to (18),
wherein the soft portion has a cylindrical member having an insulating property, a cylindrical first member that contains metal and is inserted into the cylindrical member, and a cylindrical second member that contains metal and is inserted into the first member, and
the member includes at least one of the first member or the second member.
   (20)

The endoscope device according to (19),
wherein at least one of the first member or the second member is made of magnetizable austenitic stainless steel.
   (21)

The endoscope device according to (16), further comprising:
a magnetic detection unit disposed on the movement path,
wherein an insertion part of the endoscope has a member containing metal, which extends in a longitudinal direction and has a magnetic pattern formed along the longitudinal direction,
the magnetic detection unit detects a magnetic field from the member,
the processor derives the distance based on the magnetic field detected by the magnetic detection unit,
the insertion part has a cylindrical member having an insulating property, a cylindrical first member that contains metal and is inserted into the cylindrical member, and a cylindrical second member that contains metal and is inserted into the first member,
the member includes at least one of the first member or the second member,
the first member is a spiral tube, and
the second member is a net body.
   (22)

The endoscope device according to any of (17) to (21),
wherein the magnetic detection unit detects a first magnetic flux density in a first direction and a second magnetic flux density in a second direction intersecting the first direction, at a plurality of positions along the longitudinal direction of the member.
   (23)

A processing method comprising:
acquiring a distance from a reference position on a movement path of an endoscope to a distal end of the endoscope that is moved along the movement path;
acquiring a captured image that is captured by the endoscope; and
determining a reaching site of the distal end of the endoscope inserted into a subject, based on the captured image and the distance.

Although various embodiments have been described above, it goes without saying that the present invention is not limited to these examples. It is apparent that those skilled in the art may perceive various modification examples or correction examples within the scope disclosed in the claims, and those examples are also understood as falling within the technical scope of the present invention. In addition, each constituent in the embodiment may be used in any combination without departing from the gist of the invention.

The present application is based on Japanese Patent Application (JP2022-174971) filed on Oct. 31, 2022, the content of which is incorporated in the present application by reference.

### EXPLANATION OF REFERENCES

1: endoscope
MA, MA1, MA2: magnetic pole portion
4P: processor
4: processor device
5: light source device
6: input unit
7: display device
8: expansion device
8P: processor
10A: soft portion
10B: bendable part
10C: distal end part
10: insertion part
11: operating part
12: angle knob
13A, 13B: connector portion
13: universal cord
14: first member
15a: metal strip
15: second member
16A, 16B: cap
17N: positive pole region
17S: negative pole region
17: tubular member
18A: inner resin layer
18B: outer resin layer
18: outer skin layer
19: coating layer
40: detection unit
42: housing
42A: body part
42B: lid portion
42a: flat plate portion
42b: side wall portion
42c: inner wall portion
41A, 41B, 41: through-hole
43, 44: magnetic detection unit
45: communication chip
46: storage battery
47: power receiving coil
50A: anus
53: rectum
54: sigmoid colon
55: descending colon
56: transverse colon
57: ascending colon
58: ileocecum
50: subject
81, 82, 83: recognition model
83A: determination unit
100: endoscope device
200: endoscope system
300: magnetic field generation device
PO1, PO2: position

What is claimed is:

1. A processing device comprising:
a processor configured to:
acquire a distance from a reference position on a movement path of an endoscope to a distal end of the endoscope that is moved along the movement path;
acquire a captured image that is captured by the endoscope; and
determine a reaching site of the distal end of the endoscope inserted into a subject, based on the captured image and the distance,
wherein the reference position with which the processor is configured to acquire the distance in a first period in which the endoscope is moving from a starting point to an ending point of the movement path, and the reference position with which the processor is configured to acquire the distance in a second period in which the endoscope is moving from the ending point to the starting point of the movement path are different.

2. The processing device according to claim 1,
wherein the processor is configured to determine the reaching site further based on a change amount in the distance per unit time.

3. The processing device according to claim 2,
wherein the processor is configured to determine whether or not the reaching site is a turnaround point of the distal end of the endoscope in an examination using the endoscope, based on the captured image, the distance in a case where the reference position is a position on the movement path at a starting point side, and the change amount.

4. The processing device according to claim 3,
wherein the turnaround point includes an ileocecum.

5. The processing device according to claim 1,
wherein the reference position with which the processor is configured to acquire the distance in the second period is an ending point position of the movement path, and
the reference position with which the processor is configured to acquire the distance in the first period is a position at a starting point side of the movement path.

6. The processing device according to claim 1,
wherein the processor is configured to determine the reaching site with different processing contents between the first period and the second period.

7. The processing device according to claim 1,
wherein the processor is configured to:
perform recognition processing related to an examination using the endoscope based on the captured image; and
perform, in a case where a specific recognition result is obtained by the recognition processing, determination of the reaching site based on the captured image used in the recognition processing and the distance.

8. The processing device according to claim 7,
wherein the processor is configured to store the specific recognition result, a determination result of the reaching site, and the distance in association with one another.

9. The processing device according to claim 1,
wherein the processor is configured to determine the reaching site based on an output of a machine learning model obtained by inputting an image based on the distance and the captured image to the machine learning model.

10. The processing device according to claim 1,
wherein the processor is configured to determine the reaching site based on an output of a machine learning model obtained by inputting the captured image to the machine learning model and inputting an image based on the distance to an intermediate layer of the machine learning model.

11. The processing device according to claim 1,
wherein the processor is configured to determine the reaching site based on the distance and an output of a machine learning model obtained by inputting the captured image to the machine learning model.

12. The processing device according to claim 1,
wherein the processor is configured to store information related to an examination of the subject performed using the endoscope and a determination result of the reaching site in association with each other.

13. The processing device according to claim 1,
wherein the processor in configured to output operation support information of the endoscope based on a determination result of the reaching site.

14. The processing device according to claim 1,
wherein a magnetic pattern is formed along a longitudinal direction on an insertion part of the endoscope, and
the processor is configured to acquire the distance based on a magnetic field from the magnetic pattern, which is detected by a magnetic detector positioned outside a subject.

15. An endoscope device comprising:
the processing device according to claim 1; and
the endoscope.

16. The endoscope device according to claim 15, further comprising:
a magnetic detector disposed on the movement path,
wherein an insertion part of the endoscope has a member containing metal, which extends in a longitudinal direction and has a magnetic pattern integrally formed along the longitudinal direction,
the magnetic detector detects a magnetic field from the member, and
the processor is configured to derive the distance based on the magnetic field detected by the magnetic detector.

17. The endoscope device according to claim 16,
wherein the insertion part includes a soft portion of the endoscope.

18. The endoscope device according to claim 17,
wherein the soft portion has a cylindrical member having an insulating property, a cylindrical first member that contains metal and is inserted into the cylindrical member, and a cylindrical second member that contains metal and is inserted into the first member, and
the member containing metal includes at least one of the first member or the second member.

19. The endoscope device according to claim 18,
wherein at least one of the first member or the second member is made from magnetizable austenitic stainless steel.

20. The endoscope device according to claim 15, further comprising:
a magnetic detector disposed on the movement path,
wherein an insertion part of the endoscope has a member containing metal, which extends in a longitudinal direction and has a magnetic pattern formed along the longitudinal direction,
the magnetic detector detects a magnetic field from the member,
the processor is configured to derive the distance based on the magnetic field detected by the magnetic detector,
the insertion part has a cylindrical member having an insulating property, a cylindrical first member that contains metal and is inserted into the cylindrical member, and a cylindrical second member that contains metal and is inserted into the first member,
the member containing metal includes at least one of the first member or the second member,
the first member is a spiral tube, and
the second member is a net body.

21. The endoscope device according to claim 16,
wherein the magnetic detector detects a first magnetic flux density in a first direction and a second magnetic flux density in a second direction intersecting the first direction, at a plurality of positions along the longitudinal direction of the member.

22. A processing method comprising:
acquiring a distance from a reference position on a movement path of an endoscope to a distal end of the endoscope that is moved along the movement path;
acquiring a captured image that is captured by the endoscope; and
determining a reaching site of the distal end of the endoscope inserted into a subject, based on the captured image and the distance,
wherein the reference position with which the acquiring of the distance is performed in a first period in which the endoscope is moving from a starting point to an ending point of the movement path, and the reference position with which the acquiring of the distance is performed in a second period in which the endoscope is moving from the ending point to the starting point of the movement path are different.
